(19) United States
(12) Patent Application Publication
(10) Pub. No.: US 2018/0215797 A1
(43) Pub. Date: Aug. 2, 2018

(54) Regulatory Protein Deficient Trichoderma Cells and Methods of Use Thereof

(71) Applicant: Glykos Finland OY, Helsinki (FI)

(72) Inventors: Christopher Landowski, Helsinki (FI); Ann Westerholm-Parvinen, Kirkkonummi (FI); Markku Saloheimo, Helsinki (FI)

(73) Assignee: Glykos Finland OY, Helsinki (FI)

(21) Appl. No.: 15/751,913

(22) PCT Filed: Aug. 11, 2016
(86) PCT No.: PCT/EP2016/069101

§ 371 Date: Feb. 12, 2018

(30) Foreign Application Priority Data
Aug. 13, 2015 (EP) 15180972.0

(51) Int. Cl. C07K 14/37; C12N 1/14; C12N 9/10; C12N 15/113

(52) U.S. Cl. CPC C07K 14/37; C12N 1/14; C12N 9/1051; C12N 2310/14; C12Y 204/01149; C12N 15/1137; C12Y 204/01258

(57) ABSTRACT

The present disclosure relates to compositions and methods useful for the production of heterologous proteins in filamentous fungal cells.

### FIELD OF THE INVENTION

The present disclosure relates to compositions and methods useful for the production of heterologous proteins in filamentous fungal cells, and more specifically in Trichoderma cells.

### BACKGROUND

Posttranslational modification of eukaryotic proteins, particularly therapeutic proteins such as immunoglobulins, is often necessary for proper protein folding and function. Because standard prokaryotic expression systems lack the proper machinery necessary for such modifications, alternative expression systems have to be used in production of these therapeutic proteins. Even where eukaryotic proteins do not have posttranslational modifications, prokaryotic expression systems often lack necessary chaperone proteins required for proper folding. Yeast and fungi are attractive options for expressing proteins as they can be easily grown at a large scale in simple media, which allows low production costs, and yeast and fungi have posttranslational machinery and chaperones that perform similar functions as found in mammalian cells. Moreover, tools are available to manipulate the relatively simple genetic makeup of yeast and fungal cells as well as more complex eukaryotic cells such as mammalian or insect cells (De Pourcq et al., Appl Microbiol Biotechnol, 87(5):1617-31). Despite these advantages, many therapeutic proteins are still being produced in mammalian cells, which produce therapeutic proteins with posttranslational modifications most resembling the native human proteins, whereas the posttranslational modifications naturally produced by yeast and fungi often differ from that found in mammalian cells.

To address this deficiency, new strains of yeast and fungi are being developed that produce posttranslational modifications that more closely resemble those found in native human proteins. Thus, there has been renewed interest in using yeast and fungal cells to express more complex proteins. However, due to the industry's focus on mammalian cell culture technology for such a long time, the fungal cell expression systems such as Trichoderma are not as well established as mammalian cell culture and therefore suffer from drawbacks when expressing mammalian proteins.

WO2013/102674 and WO2015/004241 discloses protease deficient filamentous fungal cells and their use for producing heterologous proteins. The reduction of certain protease activity has indeed been shown to be correlated to higher expression yield of heterologous polypeptide in those protease deficient filamentous fungal cells.

A need remains in the art for still further improved filamentous fungal cells, such as Trichoderma fungus cells, that can stably produce heterologous proteins, such as immunoglobulins, preferably at high levels of expression.

### SUMMARY

Described herein are Trichoderma cells having reduced or no activity in one or more regulatory proteins selected from the group consisting of ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), ptf7 (SEQ ID NO:11) and prp2 (SEQ ID NO:12). For example, said Trichoderma cell is Trichoderma reesei.

In a specific embodiment, said Trichoderma cell comprises a mutation in at least one gene encoding said regulatory protein selected from prp1, prp2, ptf1, ptf2, ptf3, ptf4, ptf5, ptf6, ptf7, ptf8, ptf9 and ptf10, said mutation rendering said regulatory protein non-functional.

In a specific embodiment that may be combined with the previous embodiments, said Trichoderma cell is selected from the group consisting of Δprp1, Δptf1, Δprp1 Δptf1, Δptf2, Δptf3, Δptf4, Δptf4 Δprp1 Δptf1, Δptf7, Δptf7 Δprp1 Δptf1, Δptf9, Δptf9 Δprp1 Δptf1, Δptf8 and Δptf8 Δprp1 Δptf1 deletion mutant Trichoderma cells.

In another specific embodiment that may be combined with any of the previous embodiments, the Trichoderma cell comprises a mutation in at least one gene encoding a protease, said mutation reducing or eliminating the corresponding protease activity, and said protease being selected from the group consisting of pep1, tsp1, slp1, gap1, gap2, pep4, pep3, and pep5.

In another specific embodiment that may be combined with any of the previous embodiments, the Trichoderma cell comprises a mutation in at least one gene encoding a protease, said mutation reducing or eliminating the corresponding protease activity, and said protease being selected from the group consisting of pep4, pep8, pep9, pep11, slp5, cpa5, cpa2, cpa3, amp3, tpp1, pep12, amp2, mp1, mp2, mp3, mp4, mp5, amp1, sep1, slp2, slp3, slp6, slp7 and slp8.

In another specific embodiment that may be combined with any of the previous embodiments, the Trichoderma cell may further have reduced or no activity of ALG3 protein, in particular a mutation in a gene encoding ALG3 protein that reduces or eliminates the corresponding activity.

In another specific embodiment that may be combined with any of the previous embodiments, the Trichoderma cell may further comprise a first polynucleotide encoding the N-acetylglucosaminyltransferase I catalytic domain and, optionally, a second polynucleotide encoding the N-acetylglucosaminyltransferase II catalytic domain.

In another specific embodiment that may be combined with any of the previous embodiments, the Trichoderma cell may further comprise a polynucleotide encoding an α-1,2-mannosidase, a mannosidase II, a galactosyl transferase and/or GDP-fucose synthesis activity.

In another specific embodiment that may be combined with any of the previous embodiments, the Trichoderma cell may further comprise a recombinant nucleic acid encoding a heterologous polypeptide. Typically, said heterologous polypeptide may be a mammalian polypeptide, for example, the mammalian polypeptide is glycosylated. In specific embodiments, the mammalian polypeptide is selected from the group consisting of an antibody and their antigen-binding fragments, a growth factor, an interferon, a cytokine, and an interleukin. In other specific embodiments, the mammalian polypeptide is selected from the group consisting of insulin-like growth factor 1 (IGF1), human growth hormone (hGH), and interferon alpha 2b (IFNα2b).

It is further disclosed a method of improving heterologous polypeptide production in a Trichoderma cell expression system, comprising
a. providing a Trichoderma cell as described above in which one or more regulatory proteins have reduced or eliminated activity, and
b. culturing said Trichoderma cell for production of a heterologous polypeptide. The heterologous polypeptide is advantageously produced at a higher yield when compared to the heterologous polypeptide produced in a corresponding parental Trichoderma cell in which said one or more regulatory proteins do not have reduced or eliminated activity.

The invention also relates to a method of making a heterologous polypeptide, comprising
a. providing a Trichoderma cell as defined above;
b. culturing said Trichoderma cell for production and secretion of a heterologous polypeptide in the culture medium; and,
c. recovering and, optionally, purifying the heterologous polypeptide from the culture medium.

In a specific embodiment of such method of making a heterologous polypeptide, the expression is reduced by contacting the cell with siRNA compounds directed against one or more of the genes encoding said regulatory proteins.

Thus, it is also disclosed a siRNA compound directed against a gene encoding a regulatory protein selected from the group consisting of ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), ptf7 (SEQ ID NO:11) and prp2 (SEQ ID NO:12).

In one specific embodiment, the siRNA compound may be further directed to a gene encoding a protease. In specific embodiments, said protease is selected from the group consisting of pep4, pep8, pep9, pep11, slp5, cpa5, cpa2, cpa3, amp3, tpp1, pep12, amp2, mp1, mp2, mp3, mp4, mp5, amp1, sep1, slp2, slp3, slp6, slp7, slp8.

### DESCRIPTION OF THE FIGURES

FIG. 1 depicts a map of a RNA hairpin vector containing protease regulator gene target sequences.

FIG. 2 depicts small RNA hairpin combination vector with protease and protease regulatory gene target sequences.

### DETAILED DESCRIPTION

The present invention relates to improved methods of producing recombinant heterologous polypeptides in filamentous fungal cells that have reduced or no activity in one or more of certain regulatory proteins. The present invention is based in part upon the surprising discovery that reducing the activity of certain regulatory proteins in filamentous fungal cells correlated with reduced protease activity and an increase in the expression and stability of a variety of recombinantly expressed heterologous proteins, such as immunoglobulins and growth factors.

In particular, the inventors have confirmed that either deleting the genes responsible for the particular regulatory proteins, or reducing the expression of said genes by siRNA compounds, achieved a significant reduction in certain protease activity, which correlates to a significant increase of heterologous polypeptide production in such Trichoderma cells containing such deletions, or cultured with such siRNA.

### Definitions

As used herein, an “immunoglobulin” refers to a multimeric protein containing a heavy chain and a light chain covalently coupled together and capable of specifically combining with antigen Immunoglobulin molecules are a large family of molecules that include several types of molecules such as IgM, IgD, IgG, IgA, and IgE.

As used herein, an “antibody” refers to intact immunoglobulin molecules, as well as fragments thereof which are capable of binding an antigen. These include hybrid (chimeric) antibody molecules (see, e.g., Winter et al. Nature 349:293-99225, 1991; and U.S. Pat. No. 4,816,567 226); F(ab′)2 and F(ab) fragments and Fv molecules; non-covalent heterodimers [227, 228]; single-chain Fv molecules (scFv) (see, e.g., Huston et al. Proc. Natl. Acad. Sci. U.S.A. 85:5897-83, 1988); dimeric and trimeric antibody fragment constructs; minibodies (see, e.g., Pack et al. Biochem 31, 1579-84, 1992; and Cumber et al. J. Immunology 149B, 120-26, 1992); humanized antibody molecules (see e.g., Riechmann et al. Nature 332, 323-27, 1988; Verhoeyan et al. Science 239, 1534-36, 1988; and GB 2,276,169); and any functional fragments obtained from such molecules, as well as antibodies obtained through non-conventional processes such as phage display. Preferably, the antibodies are monoclonal antibodies. Methods of obtaining monoclonal antibodies are well known in the art.

As used herein, a “peptide” and a “polypeptide” are amino acid sequences including a plurality of consecutive polymerized amino acid residues. For purpose of this invention, typically, peptides are those molecules including up to 50 amino acid residues, and polypeptides include more than 50 amino acid residues. The peptide or polypeptide may include modified amino acid residues, naturally occurring amino acid residues not encoded by a codon, and non-naturally occurring amino acid residues. As used herein, “protein” may refer to a peptide or a polypeptide of any size.

### Regulatory Proteins of the Disclosure

It is herein disclosed filamentous fungal cells, such as Trichoderma fungal cells, that can be used as expression system enabling high yield production of a heterologous polypeptide, such as a mammalian polypeptide, characterized in that they have reduced or no detectable activity in one or more regulatory proteins selected from ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), ptf7 (SEQ ID NO:11), and prp2 (SEQ ID NO:12).

A high expression of such regulatory proteins has been shown to correlate with a higher total protease activity, together with higher expression of certain proteases in Trichoderma cell. Thus, by reducing or eliminating the activity of such regulatory proteins in filamentous fungal cells, for example, in Trichoderma cells, that produce a heterologous polypeptide, the stability of the produced polypeptide is increased, resulting in an increased level of production of the polypeptide, and in some circumstances, improved quality of the produced polypeptide (e.g., full-length instead of degraded).

The regulatory proteins as found in wild type Trichoderma cells are described in more detail in the following Table 1:

| | | Protein | Gene |
|---|---|---|---|
| Gene # | Gene name | SEQ ID NO: | SEQ ID NO: |
| tre3449 | Ptf1 | 1 | 13 |
| tre122069 | Prp1 | 2 | 14 |
| tre108940 | Ptf9 | 3 | 15 |
| tre59740 | Ptf3 | 4 | 16 |
| tre106706 | Ptf8 | 5 | 17 |
| tre103158 | Ptf5 | 6 | 18 |
| tre103275 | Ptf6 | 7 | 19 |
| tre105269 | Ptf2 | 8 | 20 |
| tre76505 | Ptf4 | 9 | 21 |
| tre121130 | Ptf10 | 10 | 22 |
| tre106259 | Ptf7 | 11 | 23 |
| tre102947 | Prp2 | 12 | 24 |

### Methods of Reducing the Activity of the Regulatory Proteins of the Disclosure

Further aspects of the present disclosure relate to reducing or eliminating the activity of proteases found in filamentous fungal cells, for example Trichoderma cells, and more specifically a Trichoderma cell that produces a heterologous polypeptide, such as a mammalian polypeptide. In particular, the methods comprises reducing or eliminating the activity of one or more of the regulatory proteins of Trichoderma cells as described in Table 1 above or their corresponding homologous proteins in other related filamentous fungal species.

The activity of the regulatory proteins can be reduced in a filamentous fungal cell by any method known to those of the skilled person in the art.

In some embodiments reduced activity of a regulatory protein is achieved by reducing the expression of the corresponding gene, for example, by promoter modification of the corresponding gene or RNAi directed against the corresponding mRNA.

In other embodiments, reduced activity of the regulatory proteins is achieved by modifying the gene encoding the regulatory protein in such filamentous fungal cell, e.g. a Trichoderma cell, more specifically to disrupt or delete essential part of the gene and render the resulting mutant protein non-functional. Examples of such modifications include, without limitation, a knock-out mutation, a truncation mutation, a point mutation, a missense mutation, a substitution mutation, a frameshift mutation, an insertion mutation, that results in a reduction or elimination of the corresponding regulatory protein activity. Methods of generating at least one mutation in a regulatory protein encoding gene of interest are well known in the art and include, without limitation, random mutagenesis and screening, site-directed mutagenesis, PCR mutagenesis, insertional mutagenesis, chemical mutagenesis, and irradiation.

In certain embodiments, a portion of the regulatory protein encoding gene is modified, such as the region encoding the DNA binding domain or activating domain of a transcription factor, the coding region, or a control sequence required for expression of the coding region are deleted. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, without limitation, a leader sequence, a propeptide sequence, a signal sequence, a transcription terminator, and a transcriptional activator.

Regulatory protein encoding genes of the present disclosure may also be modified by utilizing gene deletion techniques to reduce or eliminate expression of said gene. Gene deletion techniques enable the partial or complete removal of the gene thereby eliminating their expression. In such methods, deletion of the gene may be accomplished by homologous recombination using a plasmid that has been constructed to contiguously contain the 5′ and 3′ regions flanking the gene.

The regulatory protein encoding genes of the present disclosure may also be modified by introducing, substituting, and/or removing one or more nucleotides in the gene, or a control sequence thereof required for the transcription or translation of the gene. For example, nucleotides may be inserted or removed for the introduction of a stop codon, the removal of the start codon, or a frame-shift of the open reading frame. Such a modification may be accomplished by methods known in the art, including without limitation, site-directed mutagenesis and PCR generated mutagenesis (see, for example, Botstein and Shortie, 1985, Science 229: 4719; Lo et al., 1985, Proceedings of the National Academy of Sciences USA 81: 2285; Higuchi et al., 1988, Nucleic Acids Research 16: 7351; Shimada, 1996, Meth. Mol. Bioi. 57: 157; Ho et al., 1989, Gene 77: 61; Horton et al., 1989, Gene 77: 61; and Sarkar and Sommer, 1990, BioTechniques 8: 404).

Additionally, regulatory protein encoding genes of the present disclosure may be modified by gene disruption techniques by inserting into the gene a disruptive nucleic acid construct containing a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a nonfunctional gene product results. A disrupting construct may be simply a selectable marker gene accompanied by 5′ and 3′ regions homologous to the gene. The selectable marker enables identification of transformants containing the disrupted gene.

Regulatory protein encoding genes of the present disclosure may also be modified by the process of gene conversion (see, for example, Iglesias and Trautner, 1983, Molecular General Genetics 189:5 73-76). For example, in the gene conversion a nucleotide sequence corresponding to the gene is mutagenized in vitro to produce a defective nucleotide sequence, which is then transformed into a Trichoderma strain to produce a defective gene. By homologous recombination, the defective nucleotide sequence replaces the endogenous gene. It may be desirable that the defective nucleotide sequence also contains a marker for selection of transformants containing the defective gene.

Regulatory protein encoding genes of the present disclosure may also be modified by established anti-sense techniques using a nucleotide sequence complementary to the nucleotide sequence of the gene (see, for example, Parish and Stoker, 1997, FEMS Microbiology Letters 154: 151-157). In particular, expression of the gene by filamentous fungal cells may be reduced or inactivated by introducing a nucleotide sequence complementary to the nucleotide sequence of the gene, which may be transcribed in the strain and is capable of hybridizing to the mRNA produced in the cells. Under conditions allowing the complementary anti-sense nucleotide sequence to hybridize to the mRNA, the amount of protein translated is thus reduced or eliminated.

Regulatory protein encoding genes of the present disclosure may also be modified by random or specific mutagenesis using methods well known in the art, including without limitation, chemical mutagenesis (see, for example, Hopwood, The Isolation of Mutants in Methods in Microbiology (J. R. Norris and D. W. Ribbons, eds.) pp. 363-433, Academic Press, New York, 25 1970). Modification of the gene may be performed by subjecting filamentous fungal cells to mutagenesis and screening for mutant cells in which expression of the gene has been reduced or inactivated. The mutagenesis, which may be specific or random, may be performed, for example, by use of a suitable physical or chemical mutagenizing agent, use of a suitable oligonucleotide, subjecting the DNA sequence to peR generated mutagenesis, or any combination thereof. Examples of physical and chemical mutagenizing agents include, without limitation, ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the Trichoderma cells to be mutagenized in the presence of the mutagenizing agent of choice under suitable conditions, and then selecting for mutants exhibiting reduced or no expression of the gene.

Regulatory protein encoding genes of the present disclosure may also be modified by CRISPR-CAS system, or clustered regularly interspaced short palindromic repeats. CRISPR-Cas system is a novel technique of gene editing (silencing, enhancing or changing specific genes). By inserting a plasmid containing cas9 genes and specifically designed CRISPRs, the organism's genome can be cut at any desired location. Cas9 gene originates from the type II bacterial CRISPR system of Streptococcus pyogenes. Gene product, CAS9 nuclease, complexes with a specific genome targeting CRISPR guideRNA and has high site specificity of the DNA cutting activity. It has been shown recently that CAS9 can function as an RNA guided endonuclease in various heterologous organisms (Mali et al. 2013: RNA guided human genome engineering via Cas9. Science 339:823-826; Cong et al 2013: Multiplex genome engineering using CRISPR-Cas systems. Science 339:819-823; Jiang et al 2013: RNA-guided editing of bacterial genomes using CRISPR-Cas systems. Nat Biotechnol 31:233-239; Jinek et al. 2013: RNA programmed genome editing in human cells. eLife 2:e00471; Hwang et al. 2013: Efficient genome editing in zebrafish using a CRISPR-Cas system. Nat Biotech 31:227-279. DiCarlo et al 2013: Genome engineering in Saccharomyces cerevisiae using CRISPR-Cas systems. NAR 41:4336-4343). Also filamentous fungi are have been modified with CRISPR-Cas system (Arazoef et al. (2015) Tailor-made CRISPR/Cas system for highly efficient targeted gene replacement in the rice blast fungus. Biotechnol Bioeng. doi: 10.1002/bit.25662). GuideRNA synthesis have been usually carried out from promoters transcribed by RNA polymerase III, most commonly used being SNR52 snoRNA promoter in yeasts and U3/U6 snoRNA promoters in plants and animals. Promoters transcribed by RNA polymerase II have been considered to be unsuitable for guideRNA synthesis because of the posttranscriptional modifications, 5′capping, 5′/3′ UTR's and poly A tailing. However, it has been recently demonstrated that RNA polymerase II type promoters can be used if the guideRNA sequence is flanked with self-processing ribozyme sequences. Primary transcript then undergoes self-catalyzed cleavage and generates desired gRNA sequence (Gao and Zhao 2014: Self processing of ribozyme-flanked RNAs into guide RNA's in vitro and in vivo for CRISPR-mediated genome editing. Journal of Integrative Plant Biology epublication ahead of print; March 2014).

In another specific embodiment, the activity of a regulatory protein of the present disclosure may also be reduced by established RNA interference (RNAi) techniques (see, for example, WO 2005/056772 and WO 2008/080017) and for example by culturing the filamentous fungal cell, e.g. a Trichoderma cell, under the presence of an efficient concentration of a short interference RNA (siRNA) compound directed against said regulatory protein encoding gene, as described below, and thereby reducing the activity of said regulatory protein in said filamentous fungal cell.

In certain embodiments, the at least one mutation or modification in a regulatory protein encoding gene of the present disclosure results in a modified total protease activity. In other embodiments, the at least one modification in a regulatory protein encoding gene of the present disclosure results in a decreased protease activity in one or more of the following protease activity: pep4, pep8, pep9, pep11, slp5, cpa2, cpa3, and amp3.

In certain embodiments, for example, in a Trichoderma cell, the at least one mutation or modification in a regulatory protein encoding gene of the present disclosure results in a reduction of total protease activity to 90%, 80%, 70%, 60%, 50% or less, as compared to the total protease activity in the corresponding parental Trichoderma cell with no mutation or modification in said regulatory protein encoding gene.

### Filamentous Fungal Cells of the Disclosure

It is further disclosed herein filamentous fungal cells, such as Trichoderma cells, having reduced or no activity in one or more regulatory proteins as described in Table 1, and their use for producing heterologous polypeptides, such as mammalian polypeptides.

“Filamentous fungal cells” include cells from all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). Filamentous fungal cells are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

Any filamentous fungal cell may be used in the present disclosure so long as it remains viable after being transformed with a sequence of nucleic acids and/or being modified or mutated to decrease protease activity. Preferably, the filamentous fungal cell is not adversely affected by the transduction of the necessary nucleic acid sequences, the subsequent expression of the proteins (e.g., mammalian proteins), or the resulting intermediates.

Examples of suitable filamentous fungal cells include, without limitation, cells from an Acremonium, Aspergillus, Fusarium, Humicola, Mucor, Myceliophthora, Neurospora, Penicillium, Scytalidium, Thielavia, Tolypocladium, or Trichoderma strain. In certain embodiments, the filamentous fungal cell is from a Trichoderma sp., Acremonium, Aspergillus, Aureobasidium, Cryptococcus, Chrysosporium, Chrysosporium lucknowense, Filibasidium, Fusarium, Gibberella, Magnaporthe, Mucor, Myceliophthora, Myrothecium, Neocallimastix, Neurospora, Paecilomyces, Penicillium, Piromyces, Schizophyllum, Talaromyces, Thermoascus, Thielavia, or Tolypocladium strain.

Aspergillus fungal cells of the present disclosure may include, without limitation, Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus oryzae, or Aspergillus terreus.

Neurospora fungal cells of the present disclosure may include, without limitation, Neurospora crassa.

In certain embodiments, the filamentous fungal cell is selected from the group consisting of Trichoderma (T. reesei), Neurospora (N. crassa), Penicillium (P. chrysogenum), Aspergillus (A. nidulans, A. niger and A. oryzae), Myceliophthora (M. thermophila) and Chrysosporium (C. lucknowense).

In certain embodiments, the filamentous fungal cell is a Trichoderma fungal cell. Trichoderma fungal cells of the present disclosure may be derived from a wild-type Trichoderma strain or a mutant thereof. Examples of suitable Trichoderma fungal cells include, without limitation, Trichoderma harzianum, Trichoderma koningii, Trichoderma longibrachiatum, Trichoderma reesei, Trichoderma atroviride, Trichoderma vixens, Trichoderma viride; and alternative sexual form thereof (i.e., Hypocrea). In a specific embodiment, the filamentous fungal cell for use according to the disclosure is Trichoderma reesei.

General methods to disrupt genes of and cultivate filamentous fungal cells are disclosed, for example, for Penicillium, in Kopke et al. (2010) Application of the Saccharomyces cerevisiae FLP/FRT recombination system in filamentous fungi for marker recycling and construction of knockout strains devoid of heterologous genes. Appl Environ Microbiol. 76(14):4664-74. doi: 10.1128/AEM.00670-10, for Aspergillus, in Maruyama and Kitamoto (2011), Targeted Gene Disruption in Koji Mold Aspergillus oryzae, in James A. Williams (ed.), Strain Engineering: Methods and Protocols, Methods in Molecular Biology, vol. 765, DOI 10.1007/978-1-61779-197-0_27; for Neurospora, in Collopy et al. (2010) High-throughput construction of gene deletion cassettes for generation of Neurospora crassa knockout strains. Methods Mol Biol. 2010; 638:33-40. doi: 10.1007/978-1-60761-611-5_3; and for Myceliophthora or Chrysosporium PCT/NL2010/000045 and PCT/EP98/06496.

A method to transform filamentous fungal cells include Agrobacterium mediated transformation. Gene transformation method based on Agrobacterium tumefaciens T-DNA transfer to host cell has been originally developed with plants. It has been applied to yeasts (Bundock et al. (1995) EMBO J 14:3206-3214) and filamentous fungi (de Groot et al. (1998) Nat Biotechnol 16:839-842). If the T-DNA includes homologous regions with fungal genome, the integration to host cell can occur through homologous recombination, thus, enabling targeted knockouts and gene replacements (Gouka et al. (1999) Nat Biotechnol 17:598-601) (Zeilinger (2004) Curr Genet 45:54-60) (Zwiers and De Waard (2001) Curr Genet 39:388-393) (Zhang et al. (2003) Mol Gen Genomics 268:645-655). In general, the expression cassette with gene of interest and promoter/terminator sequences functional in fungal host can be flanked with sequences homologus to the regions flanking the sequence to be knocked out from fungal genome. Cassette with homologous flanks is then inserted to Agrobacterium tumefaciens binary vector between the T-DNA borders, left border and right border. Binary vector can be electroporated to Agrobacterium tumefaciens strain like C58C1 pGV2260 or LBA pAL4404 containing the helper plasmid encoding vir proteins needed for T-DNA transfer. Co-cultivation of Trichoderma reesei and Agrobacterium can be made by mixing the fungal spores or pre-germinated spores or protoplasts with Agrobacterium suspension culture and plating the mixture to sterile cellophane disks placed on top of the transformation plates. On the absence of wounded plant tissue, vir-gene induction can be launched by the presence of inducing agents in the culture media, like asetosyringone. After of two days of co-cultivation, sellophane disks can be transferred on top of selection plates, containing the selective agent for transformed Trichoderma cells and an antibiotic agent inhibiting the Agrobacterium growth with no adverse effects on Trichoderma, like ticarcillin. Once the transformed fungal colonies appear, they can be picked and purified through single spore cultures, as routinely done with other transformation methods.

Certain aspects of the present disclosure relate to filamentous fungal cells, for example a Trichoderma cell having reduced or no detectable activity in one or more regulatory proteins selected from ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), prp2 (SEQ ID NO:12), and ptf7 (SEQ ID NO:11).

Certain aspects of the present disclosure relate to filamentous fungal cells, for example, Aspergillus cells, having reduced or no detectable activity in one or more regulatory proteins selected from EHA27990.1 (SEQ ID NO:187), XP_001389638.1 (SEQ ID NO:188), XP_001401764.1 (SEQ ID NO:204), EHA21595.1 (SEQ ID NO:189), XP_001398220.2 (SEQ ID NO:190) and XP_001388587.2 (SEQ ID NO:191).

Certain aspects of the present disclosure relate to filamentous fungal cells, for example, Neurospora cells, having reduced or no detectable activity in one or more regulatory proteins selected from XP_958054.1 (SEQ ID NO:192), XP_964115.2 (SEQ ID NO:193), XP_965444.2 (SEQ ID NO:194), XP_961139.2 (SEQ ID NO:195), CAC28684.1 (SEQ ID NO:196), and XP_011392968.1 (SEQ ID NO:197).

Certain aspects of the present disclosure relate to filamentous fungal cells, for example, Myceliophthora cells, having reduced or no detectable activity in one or more regulatory proteins selected from XP_003661571.1 (SEQ ID NO:198), XP_003659580.1 (SEQ ID NO:199), and XP_003658871.1 (SEQ ID NO:200).

Certain aspects of the present disclosure relate to filamentous fungal cells, for example, Fusarium cells, having reduced or no detectable activity in EWG43214.1 (SEQ ID NO:201).

Certain aspects of the present disclosure relate to filamentous fungal cells, for example, Penicillium cells, having reduced or no detectable activity in one or more regulatory proteins selected from CDM30613.1 (SEQ ID NO:202) and XP_002567858.1 (SEQ ID NO:203).

Other aspects of the present disclosure relate to a Trichoderma fungal cell, which comprises a mutation, for example a gene deletion, in at least one gene encoding said regulatory protein selected from prp1, prp2, ptf1, ptf2, ptf3, ptf4, ptf5, ptf6, ptf7, ptf8, ptf9 and ptf10, said mutation rendering said regulatory protein non-functional.

In a specific embodiment, the Trichoderma cell is selected from the group consisting of Δprp1, Δptf1, Δprp1 Δptf1, Δptf2, Δptf3, Δptf4, Δptf4 Δprp1 Δptf1, Δptf7, Δptf7 Δprp1 Δptf1, Δptf9, Δptf9 Δprp1 Δptf1, Δptf8 and Δptf8 Δprp1 Δptf1 deletion mutant Trichoderma cells.

Combinations of Mutations in Regulatory Protein Encoding Genes and/or Protease Encoding Genes

The filamentous fungal cells or Trichoderma fungal cells of the present disclosure may contain reduced activity in a combination of those regulatory proteins selected from the group consisting of ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), prp2 (SEQ ID NO:12), and ptf7 (SEQ ID NO:11).

In some embodiments, the filamentous fungal cell, for example, a Trichoderma cell has reduced or no expression levels of at least the regulatory protein encoding genes prp1 and ptf1 genes. In certain embodiments, the filamentous fungal cell, for example a Trichoderma cell, has reduced or no expression levels of at least the regulatory protein encoding genes ptf4, prp1 and ptf1 genes. In other embodiments, the filamentous fungal cell, or Trichoderma cell, has reduced or no expression levels of at least the regulatory protein encoding genes ptf7, prp1, and ptf1. In other embodiments, the filamentous fungal cell, or Trichoderma cell, has reduced or no expression levels of at least the regulatory protein encoding genes ptf8, prp1, and ptf1. In other embodiments, the filamentous fungal cell, or Trichoderma cell, has reduced or no expression levels of at least the regulatory protein encoding genes ptf9, prp1, and ptf1.

For example, the filamentous fungal, typically a Trichoderma cell, is selected from the group consisting of Δprp1 Δptf1, Δptf4 Δprp1 Δptf1, Δptf7 Δprp1 Δptf1, Δptf8 Δprp1 Δptf1, Δptf9 Δprp1 Δptf1, deletion mutant Trichoderma cells.

Reduction or elimination of the regulatory proteins of the disclosure in a filamentous fungal cell, for example a Trichoderma cell, results in a decreased protease activity in one or more of the following protease activity: pep4, pep8, pep9, pep11, slp5, cpa2, cpa3, and amp3.

Advantageously, the filamentous fungal cells or Trichoderma fungal cells of the present disclosure may also have reduced activity of one or more proteases. In certain embodiments, the expression level of the one or more proteases is reduced.

Accordingly, it is hereby disclosed filamentous fungal cells or Trichoderma cells with reduced or no activity in certain regulatory proteins as described above, further comprising a mutation in at least one gene encoding a protease, said mutation reducing or eliminating the corresponding protease activity (as compared to corresponding parent strain which does not have said mutation), and said protease being selected from the group consisting of pep1, tsp1, slp1, gap1, gap2, pep4, pep3, and pep5.

It is also disclosed filamentous fungal cells or Trichoderma cells with reduced or no activity in certain regulatory proteins as described above, further comprising a mutation in at least one gene encoding a protease, said mutation reducing or eliminating the corresponding protease activity (as compared to corresponding parent strain which does not have said mutation), and said protease being selected from the group consisting of the following protease pep8, pep9, pep11, slp5, cpa5, cpa2, cpa3, amp3, tpp1, pep12, amp2, mep1, mep2, mep3, mep4, mep5, amp1, sep1, slp2, slp3, slp6, slp7, slp8.

The filamentous fungal cells or Trichoderma cells of the disclosure may further have one or more additional mutations in at least one gene encoding a protease, said mutation reducing or eliminating the corresponding protease activity (as compared to corresponding parent strain which does not have said mutation), and said protease being selected from the group consisting of
an aspartic protease pep6, pep10, pep13, pep14, or pep16;
slp like protease slp57433, slp35726, slp60791, or slp109276;
gap like protease gap3 or gap4;
sedolisin like protease sed2, sed3, or sed5;
Group A protease selected from the group of protease65735, protease77577, protease81087, protease56920, protease122083, protease79485, protease120998, or protease61127;
Group B protease selected from the group of protease21659, protease58387, protease75159, protease56853, or protease64193;
Group C protease selected from the group of protease82452, protease80762, protease21668, protease81115, protease82141, protease23475;
Group D protease selected from the group of protease121890, protease22718, protease47127, protease61912, protease80843, protease66608, protease72612, protease40199; or
Group E protease selected from the group of protease22210, protease111694, protease82577.
(the corresponding amino acid sequences of these proteases are disclosed in WO2015/004241 or available according to their ID numbers at the following link http://genome.jgi-psforg/Trire2/Trire2.home.html)

Methods for reducing or eliminating protease activity in filamentous fungal cell, and the corresponding proteases are disclosed in WO2013/102674 and WO2015/004241, which contents are incorporated herein by reference. In preferred embodiments, the mutation eliminate the corresponding protease activity, in other words, the mutation renders the corresponding protease inactive. Said mutation eliminating the corresponding protease activity may typically be a deletion mutation.

Examples of suitable proteases found in Trichoderma reesei cells are disclosed in the following table 2:

TABLE 2
Proteases of Trichoderma reesei (ID number according to Joint
Genome Institute Database, see http://genome.jgi-psf.org/Trire2/
Trire2.home.html)

| Name of the | | Amino acid | Nucleotide |
|---|---|---|---|
| Protease | Gene# | SEQ ID NO: | SEQ ID NO: |
| Pep1 | tre74156 | 41 | 42 |
| Tsp1 | tre73897 | 79 | 80 |
| Slp1 | tre51365 | 63 | 64 |
| Gap1 | tre69555 | 81 | 82 |
| Gap2 | tre106661 | 83 | 84 |
| Pep4 | tre77579 | 51 | 52 |
| Pep3 | tre121133 | 85 | 86 |
| Pep8 | tre122076 | 55 | 56 |
| Pep9 | tre79807 | 57 | 58 |
| Pep11 | tre121306 | 45 | 46 |
| Slp5 | tre64719 | 67 | 68 |
| Cpa5 | tre120998 | 87 | 88 |
| Cpa3 | tre22459 | 37 | 38 |
| Amp3 | tre23475 | 29 | 30 |
| Tpp1 | tre82623 | 59 | 60 |
| Pep12 | tre119876 | 89 | 90 |
| Amp2 | tre108592 | 91 | 92 |
| Mep1 | tre122703 | 93 | 94 |
| Mep2 | tre122576 | 95 | 96 |
| Mep3 | tre4308 | 97 | 98 |
| Mep4 | tre53343 | 99 | 100 |
| Mep5 | tre73809 | 101 | 102 |
| Amp1 | tre81070 | 27 | 28 |
| Sep1 | tre124051 | 61 | 62 |
| Slp2 | tre123244 | 103 | 104 |
| Slp3 | tre123234 | 65 | 66 |
| Slp6 | tre121495 | 69 | 70 |
| Slp7 | tre123865 | 105 | 106 |
| Slp8 | tre58698 | 71 | 72 |

Accordingly, it is also disclosed a filamentous fungal cell, for example a Trichoderma cell with reduced or no activity in one or more regulatory proteins of Table 1 as described above, and further comprising a gene deletion of at least 5, 6, 7, 8 or 9 protease encoding genes.

### Heterologous Polypeptides

By reducing or eliminating (for example by gene deletion) the activity of the regulatory proteins as described in Table 1 above, the production of heterologous polypeptides in such filamentous fungal cells may be increased. Accordingly, in one specific embodiment, the filamentous fungal cell or Trichoderma cell as disclosed herein further comprises a recombinant polynucleotide encoding a heterologous polypeptide. Advantageously, said heterologous polypeptide is produced at increased levels, for example at least two-fold increased levels, as compared to the level produced in a parental cell which does not have reduced activity in said regulatory proteins, for example a parental cell which does not have a gene deletion in said regulatory protein encoding gene.

As used herein a “heterologous polypeptide” refers to a polypeptide that is not naturally found in (i.e., endogenous) a filamentous fungal cell of the present disclosure, or that is expressed at an elevated level in a filamentous fungal cell as compared to the endogenous version of the polypeptide. In certain embodiments, the heterologous polypeptide is a mammalian polypeptide. In other embodiments, the heterologous polypeptide is a non-mammalian polypeptide.

Mammalian polypeptides of the present disclosure may be any mammalian polypeptide having a biological activity of interest. As used herein, a “mammalian polypeptide” is a polypeptide that is natively expressed in a mammal, a polypeptide that is derived from a polypeptide that is natively expressed in a mammal, or a fragment thereof. A mammalian polypeptide also includes peptides and oligopeptides that retain biological activity. Mammalian polypeptides of the present disclosure may also include two or more polypeptides that are combined to form the encoded product. Mammalian polypeptides of the present disclosure may further include fusion polypeptides, which contain a combination of partial or complete amino acid sequences obtained from at least two different polypeptides. Mammalian polypeptides may also include naturally occurring allelic and engineered variations of any of the disclosed mammalian polypeptides and hybrid mammalian polypeptides.

The mammalian polypeptide may be a naturally glycosylated polypeptide or a naturally non-glycosylated polypeptide.

Examples of suitable mammalian polypeptides include, without limitation, immunoglobulins, antibodies, antigens, antimicrobial peptides, enzymes, growth factors, hormones, interferons, cytokines, interleukins, immunodilators, neurotransmitters, receptors, reporter proteins, structural proteins, and transcription factors.

Specific examples of suitable mammalian polypeptides include, without limitation, immunoglobulins, immunoglobulin heavy chains, immunoglobulin light chains, monoclonal antibodies, hybrid antibodies, F(ab′)2 antibody fragments, F(ab) antibody fragments, Fv molecules, single-chain Fv antibodies, dimeric antibody fragments, trimeric antibody fragments, functional antibody fragments, immunoadhesins, insulin-like growth factor 1, growth hormone, insulin, interferon alpha 2b, fibroblast growth factor 21, human serum albumin, camelid antibodies and/or antibody fragments, single domain antibodies, multimeric single domain antibodies, and erythropoietin.

Other examples of suitable mammalian proteins include, without limitation, an oxidoreductase, a transferase, a hydrolase, a lyase, an isomerase, a ligase, an aminopeptidase, an amylase, a carbohydrase, a carboxypeptidase, a catalase, a glycosyltransferase, a deoxyribonuclease, an esterase, a galactosidase, a betagalactosidase, a glucosidase, a glucuronidase, a glucuronoyl esterase, a haloperoxidase, an invertase, a lipase, an oxidase, a phospholipase, a proteolytic enzyme, a ribonuclease, a urokinase, an albumin, a collagen, a tropoelastin, and an elastin.

Non-mammalian polypeptides of the present disclosure may be any non-mammalian polypeptide having a biological activity of interest. As used herein, a “non-mammalian polypeptide” is a polypeptide that is natively expressed in a non-mammalian organism, such as a fungal cell, a polypeptide that is derived from a polypeptide that is natively expressed in a non-mammal organism, or a fragment thereof. A non-mammalian polypeptide also includes peptides and oligopeptides that retain biological activity. Non-mammalian polypeptides of the present disclosure may also include two or more polypeptides that are combined to form the encoded product. Non-mammalian polypeptides of the present disclosure may further include fusion polypeptides, which contain a combination of partial or complete amino acid sequences obtained from at least two different polypeptides. Non-mammalian polypeptides may also include naturally occurring allelic and engineered variations of any of the disclosed non-mammalian polypeptides and hybrid non-mammalian polypeptides.

Examples of suitable non-mammalian polypeptides include, without limitation, aminopeptidases, amylases, carbohydrases, carboxypeptidases, catalases, cellulases, chitinases, cutinases, deoxyribonucleases, esterases, alpha-galactosidases, beta-galactosidases, glucoamylases, alpha-glucosidases, beta-glucosidases, invertases, laccases, lipases, mutanases, oxidases, pectinolytic enzymes, peroxidases, phospholipases, phytases, polyphenoloxidases, proteolytic enzymes, ribonucleases, transglutaminases and xylanases.

### Recombinant Nucleic Acid Encoding Heterologous Polypeptides

Nucleic acids encoding the heterologous polypeptides of the present disclosure are prepared by any suitable method known in the art, including, without limitation, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature [e.g., in Matteucci et al., (1980) Tetrahedron Lett 21:719-722; U.S. Pat. Nos. 4,500,707; 5,436,327; and 5,700,637]. In addition, the desired nucleic acids may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid encoding the heterologous polypeptides of the present disclosure can be incorporated into an expression vector. “Expression vector” or “vector” refers to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also includes materials to aid in achieving entry of the nucleic acid into the host cell, such as a virus, liposome, protein coating, or the like. The expression vectors contemplated for use in the present disclosure include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known in the art.

Incorporation of the individual nucleic acids may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a polynucleotide having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired polynucleotide are often cleaved with the same restriction enzyme, thereby assuring that the ends of the expression vector and the ends of the polynucleotide are complementary to each other. In addition, DNA linkers maybe used to facilitate linking of nucleic acids sequences into an expression vector.

A series of individual nucleic acids can also be combined by utilizing methods that are known in the art (e.g., U.S. Pat. No. 4,683,195).

For example, each of the desired nucleic acids can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual polynucleotides may be “spliced” together and subsequently transduced into a host cell simultaneously. Thus, expression of each of the plurality of polynucleotides is affected.

Individual polynucleotides, or “spliced” polynucleotides, are then incorporated into an expression vector. The present disclosure is not limited with respect to the process by which the polynucleotide is incorporated into the expression vector. Those of ordinary skill in the art are familiar with the necessary steps for incorporating a nucleic acid into an expression vector. A typical expression vector contains the desired nucleic acid preceded by one or more regulatory regions, along with a ribosome binding site, e.g., a nucleotide sequence that is 3-9 nucleotides in length and located 3-11 nucleotides upstream of the initiation codon in E. coli. See Shine and Dalgarno (1975) Nature 254(5495):34-38 and Steitz (1979) Biological Regulation and Development (ed. Goldberger, R. F.), 1:349-399 (Plenum, New York).

The term “operably linked” as used herein refers to a configuration in which a control sequence is placed at an appropriate position relative to the coding sequence of the nucleic acid or polynucleotide such that the control sequence directs the expression of a polypeptide.

Regulatory regions include, for example, those regions that contain a promoter and an operator. A promoter is operably linked to the desired polynucleotide, thereby initiating transcription of the polynucleotide via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter (see de Boer et al., (1983) Proc Natl Acad Sci USA 80(1):21-25). As will be appreciated by those of ordinary skill in the art, these and other expression vectors may be used in the present disclosure, and the present disclosure is not limited in this respect.

Although any suitable expression vector may be used to incorporate the desired sequences, readily available expression vectors include, without limitation: plasmids, such as pSClO1, pBR322, pBBR1MCS-3, pUR, pEX, pMR100, pCR4, pBAD24, pUC19, pRS426; and bacteriophages, such as Ml 3 phage and 2 phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

Suitable expression vectors for the purposes of the invention, including the expression of the desired heterologous polypeptide, enzyme, and one or more catalytic domains described herein, include expression vectors containing the nucleic encoding the desired heterologous polypeptide, enzyme, or catalytic domain(s) operably linked to a constitutive or an inducible promoter. Examples of particularly suitable promoters for operable linkage to such polynucleotides include promoters from the following genes: gpdA, cbh1, Aspergillus oryzae TAKA amylase, Rhizomucor miehei aspartic proteinase, Aspergillus niger neutral alpha-amylase, Aspergillus niger acid stable alpha-amylase, Aspergillus niger glucoamylase (glaA), Aspergillus awamori glaA, Rhizomucor miehei lipase, Aspergillus oryzae alkaline protease, Aspergillus oryzae triose phosphate isomerase, Aspergillus nidulans acetamidase, Aspergillus oryzae acetamidase, Fusarium oxysporum trypsin-like protease, fungal endo α-L-arabinase (abnA), fungal α-L-arabinofuranosidase A (abfA), fungal α-L-arabinofuranosidase B (abfB), fungal xylanase (xlnA), fungal phytase, fungal ATP-synthetase, fungal subunit 9 (oliC), fungal triose phosphate isomerase (tpi), fungal alcohol dehydrogenase (adhA), fungal α-amylase (amy), fungal amyloglucosidase (glaA), fungal acetamidase (amdS), fungal glyceraldehyde-3-phosphate dehydrogenase (gpd), yeast alcohol dehydrogenase, yeast lactase, yeast 3-phosphoglycerate kinase, yeast triosephosphate isomerase, bacterial α-amylase, bacterial Spo2, and SSO. Examples of such suitable expression vectors and promoters are also described in PCT/EP2011/070956, the entire contents of which is hereby incorporated by reference herein.

In embodiments where the filamentous fungal cell contains a recombinant nucleic acid encoding an immunoglobulin or antibody, the filamentous fungal cell, for example, a Trichoderma fungal cell may have reduced or no expression of one or more regulatory protein encoding genes selected from prp1, prp2, ptf1, ptf2, ptf3, ptf4, ptf5, ptf6, ptf7, ptf8 ptf9 and ptf10.

In other embodiments, the filamentous fungal cell contains a recombinant polynucleotide encoding a growth factor, interferon, cytokine, or interleukin. In embodiments where the filamentous fungal cell, for example a Trichoderma fungal cell contains a recombinant polynucleotide encoding a growth factor, interferon, cytokine, human serum albumin, or interleukin, the filamentous fungal cell may have reduced or no expression of one or more regulatory protein encoding genes selected from prp1, prp2, ptf1, ptf2, ptf3, ptf4, ptf5, ptf6, per, ptf8 ptf9 and ptf10. In certain preferred embodiments, the growth factor is IGF-1 or the interferon is interferon-α 2b.

In embodiments where the filamentous fungal cell contains a recombinant nucleic acid encoding a non-mammalian polypeptide, the non-mammalian polypeptide may be an aminopeptidase, amylase, carbohydrase, carboxypeptidase, catalase, cellulase, chitinase, cutinase, deoxyribonuclease, esterase, alpha-galactosidase, beta-galactosidase, glucoamylase, alpha-glucosidase, beta-glucosidase, invertase, laccase, lipase, mutanase, oxidase, pectinolytic enzyme, peroxidase, phospholipase, phytase, polyphenoloxidase, proteolytic enzyme, ribonuclease, transglutaminase or xylanase. In embodiments where the filamentous fungal cell contains a recombinant nucleic acid encoding a non-mammalian polypeptide, the filamentous fungal cell or Trichoderma cell may have reduced or no detectable expression of one or more regulatory protein encoding genes selected from prp1, prp2, ptf1, ptf2, ptf3, ptf4, ptf5, ptf6, ptf7, ptf8 ptf9 and ptf10.

### Additional Modifications of the Glycosylation Pathway for Mimicking Human Glycosylation Pathway

In certain embodiments, the filamentous fungal cells or Trichoderma fungal cells of the present disclosure may have additional genetic modifications in their glycosylation pathway (glycoengineering).

Methods for modifying the glycosylation pathway of filamentous fungal cells, for example, Trichoderma cells, are disclosed in WO2012/069593, WO2013/102674, WO2013/174927, WO2015/004239, WO2015/001049 and WO2015/004241.

More specifically, in certain embodiments, the filamentous fungal cells or Trichoderma fungal cells of the present disclosure may have reduced or no activity of a dolichyl-P-Man:Man(5)GlcNAc(2)-PP-dolichyl mannosyltransferase. Dolichyl-P-Man:Man(5)GlcNAc(2)-PP-dolichyl mannosyltransferase (EC 2.4.1.130) transfers an alpha-D-mannosyl residue from dolichyl-phosphate D-mannose into a membrane lipid-linked oligosaccharide. Typically, the dolichyl-P-Man:Man(5)GlcNAc(2)-PP-dolichyl mannosyltransferase enzyme is encoded by an alg3 gene. Thus, in certain embodiments, the filamentous fungal cell has reduced or no activity of ALG3, which is the activity encoded by the alg3 gene. In some embodiments, the alg3 gene contains a mutation that reduces the corresponding ALG3 activity. In certain embodiments, the alg3 gene is deleted from the filamentous fungal cell.

In other embodiments, the filamentous fungal cells or Trichoderma fungal cells of the present disclosure further contain a polynucleotide encoding an α-1,2-mannosidase. The polynucleotide encoding the α-1,2-mannosidase may be endogenous in the host cell, or it may be heterologous to the host cell. These polynucleotides are especially useful for a filamentous fungal cell expressing high-mannose glycans transferred from the Golgi to the ER without effective exo-α-2-mannosidase cleavage. The α-1,2-mannosidase may be a mannosidase I type enzyme belonging to the glycoside hydrolase family 47 (cazy.org/GH47_all.html). In certain embodiments the α-1,2-mannosidase is an enzyme listed at cazy.org/GH47_characterized.html. In particular, the α-1,2-mannosidase may be an ER-type enzyme that cleaves glycoproteins such as enzymes in the subfamily of ER α-mannosidase I EC 3.2.1.113 enzymes. Examples of such enzymes include human α-2-mannosidase 1B (AAC26169), a combination of mammalian ER mannosidases, or a filamentous fungal enzyme such as α-1,2-mannosidase (MDS1) (T. reesei AAF34579; Maras M et al J Biotech. 77, 2000, 255). For ER/Golgi expression the catalytic domain of the mannosidase is typically fused with a targeting peptide, such as HDEL, KDEL, or part of an ER or early Golgi protein, or expressed with an endogenous ER targeting structures of an animal or plant mannosidase I enzyme, see, for example, Callewaert et al. 2001 Use of HDEL-tagged Trichoderma reesei mannosyl oligosaccharide 1,2-a-D-mannosidase for N-glycan engineering in Pichia pastoris. FEBS Lett 503: 173-178.

In further embodiments, the filamentous fungal cells or Trichoderma fungal cells of the present disclosure also contain an N-acetylglucosaminyltransferase I catalytic domain and an N-acetylglucosaminyltransferase II catalytic domain. Such catalytic domains are useful for expressing complex N-glycans in non-mammalian cells. N-acetylglucosaminyltransferase I (GlcNAc-TI; GnTI; EC 2.4.1.101) catalyzes the reaction UDP-N-acetyl-D-glucosamine+3-(alpha-D-mannosyl)-beta-D-mannosyl-R<=>UDP+3-(2-(N-acetyl-beta-D-glucosaminyl)-alpha-D-mannosyl)-beta-D-mannosyl-R, where R represents the remainder of the N-linked oligosaccharide in the glycan acceptor. An N-acetylglucosaminyltransferase I catalytic domain is any portion of an N-acetylglucosaminyltransferase I enzyme that is capable of catalyzing this reaction. N-acetylglucosaminyltransferase II (GlcNAc-TII; GnTII; EC 2.4.1.143) catalyzes the reaction UDP-N-acetyl-D-glucosamine+6-(alpha-D-mannosyl)-beta-D-mannosyl-R<=>UDP+6-(2-(N-acetyl-beta-D-glucosaminyl)-alpha-D-mannosyl)-beta-D-mannosyl-R, where R represents the remainder of the N-linked oligosaccharide in the glycan acceptor. An N-acetylglucosaminyltransferase II catalytic domain is any portion of an N-acetylglucosaminyltransferase II enzyme that is capable of catalyzing this reaction. Examples of suitable N-acetylglucosaminyltransferase I catalytic domains and an N-acetylglucosaminyltransferase II catalytic domains can be found in International Patent Application No. PCT/EP2011/070956. The N-acetylglucosaminyltransferase I catalytic domain and N-acetylglucosaminyltransferase II catalytic domain can be encoded by a single polynucleotide. In certain embodiments, the single polynucleotide encodes a fusion protein containing the N-acetylglucosaminyltransferase I catalytic domain and the N-acetylglucosaminyltransferase II catalytic domain. Alternatively, the N-acetylglucosaminyltransferase I catalytic domain can be encoded by a first polynucleotide and the N-acetylglucosaminyltransferase II catalytic domain can be encoded by a second polynucleotide.

In embodiments where, the filamentous fungal cell or Trichoderma fungal cell contains an N-acetylglucosaminyltransferase I catalytic domain and an N-acetylglucosaminyltransferase II catalytic domain, the cell can also contain a polynucleotide encoding a mannosidase II. Mannosidase II enzymes are capable of cleaving Man5 structures of GlcNAcMan5 to generate GlcNAcMan3, and if combined with action of a catalytic domain of GnTII, to generate G0; and further, with action of a catalytic domain of a galactosyltransferase, to generate G1 and G2. In certain embodiments mannosidase II-type enzymes belong to glycoside hydrolase family 38 (cazy.org/GH38_all.html). Examples of such enzymes include human enzyme AAC50302, D. melanogaster enzyme (Van den Elsen J. M. et al (2001) EMBO J. 20: 3008-3017), those with the 3D structure according to PDB-reference 1HTY, and others referenced with the catalytic domain in PDB. For ER/Golgi expression, the catalytic domain of the mannosidase is typically fused with an N-terminal targeting peptide, for example using targeting peptides listed in the International Patent Application No. PCT/EP2011/070956. After transformation with the catalytic domain of a mannosidase II-type mannosidase, a strain effectively producing GlcNAc2Man3, GlcNAc1Man3 or G0 is selected.

In certain embodiments that may be combined with the preceding embodiments, the filamentous fungal cell further contains a polynucleotide encoding a UDP-GlcNAc transporter.

In certain embodiments that may be combined with the preceding embodiments, the filamentous fungal cell further contains a polynucleotide encoding a β-1,4-galactosyltransferase. Generally, β-1,4-galactosyltransferases belong to the CAZy glycosyltransferase family 7 (cazy.org/GT7_all.html). Examples of useful β4GalT enzymes include β4GalT1, e.g. bovine Bos taurus enzyme AAA30534.1 (Shaper N. L. et al Proc. Natl. Acad. Sci. U.S.A. 83 (6), 1573-1577 (1986)), human enzyme (Guo S. et al. Glycobiology 2001, 11:813-20), and Mus musculus enzyme AAA37297 (Shaper, N. L. et al. 1998 J. Biol. Chem. 263 (21), 10420-10428). In certain embodiments of the invention where the filamentous fungal cell contains a polynucleotide encoding a galactosyltransferase, the filamentous fungal cell also contains a polynucleotide encoding a UDP-Gal 4 epimerase and/or UDP-Gal transporter. In certain embodiments of the invention where the filamentous fungal cell contains a polynucleotide encoding a galactosyltransferase, lactose may be used as the carbon source instead of glucose when culturing the host cell. The culture medium may be between pH 4.5 and 7.0 or between 5.0 and 6.5. In certain embodiments of the invention where the filamentous fungal cell contains a polynucleotide encoding a galactosyltransferase and, optionally, a polynucleotide encoding a UDP-Gal 4 epimerase and/or UDP-Gal transporter, a divalent cation such as Mn2+, Ca2+ or Mg2+ may be added to the cell culture medium.

In certain embodiments that may be combined with the preceding embodiments, the level of activity of alpha-1,6-mannosyltransferase in the host cell is reduced or eliminated compared to the level of activity in a wild-type host cell. In certain embodiments, the filamentous fungal has a reduced level of (or no) expression of an och1 gene compared to the level of expression in a wild-type filamentous fungal cell.

In certain embodiments, glycosyltransferases, or for example, GnTI, GnTII, or GalT or glycosylhydrolases, for example, α-1,2-mannosidase or mannosidase II, include a targeting peptide linked to the catalytic domains. The term “linked” as used herein means that two polymers of amino acid residues in the case of a polypeptide or two polymers of nucleotides in the case of a polynucleotide are either coupled directly adjacent to each other or are within the same polypeptide or polynucleotide but are separated by intervening amino acid residues or nucleotides. A “targeting peptide”, as used herein, refers to any number of consecutive amino acid residues of the recombinant protein that are capable of localizing the recombinant protein to the endoplasmic reticulum (ER) or Golgi apparatus (Golgi) within the filamentous fungal cell. The targeting peptide may be N-terminal or C-terminal to the catalytic domains. In certain embodiments, the targeting peptide is N-terminal to the catalytic domains. In certain embodiments, the targeting peptide provides direct binding to the ER or Golgi membrane. Components of the targeting peptide may come from any enzyme that normally resides in the ER or Golgi apparatus. Such enzymes include mannosidases, mannosyltransferases, glycosyltransferases, Type 2 Golgi proteins, and MNN2, MNN4, MNN6, MNN9, MNN10, MNS1, KRE2, VAN1, and OCH1 enzymes. Suitable targeting peptides are described in WO2013/102674. In one embodiment, the targeting peptide of GnTI or GnTII is human GnTII enzyme. In other embodiments, targeting peptide is derived from Trichoderma Kre2, Kre2-like, Och1, Anp1, and Van1.

In a specific embodiment, the filamentous fungal cell, for example a Trichoderma cell of the present disclosure, further comprises a polynucleotide encoding an α1,2 mannosidase, a mannosidase II, a galactosyltransferase, α1,6 fucosyltransferase, and/or GDP fucose synthesis activity.

Polynucleotides encoding GDP fucose synthesis activity includes GMD polynucleotide or a functional variant polynucleotide encoding a polypeptide having GDP-mannose-dehydratase activity; and, FX polynucleotide or a functional variant polynucleotide encoding a polypeptide having both GDP-keto-deoxy-mannose-epimerase and GDP-keto-deoxy-galactose-reductase activities. Optionally; polynucleotides encoding GDP fucose synthesis activity further includes GDP fucose transporter activity.

Polynucleotides encoding GPD fucose synthesis and α1,6 fucosyltransferase are disclosed in WO2013/174927.

### Heterologous Polypeptide Production

A heterologous polypeptide of interest is produced by filamentous fungal cells of the present disclosure having reduced or no activity of one or more of the regulatory proteins of the disclosure (e.g. of Table 1), for example by cultivating the cells in a nutrient medium for production of the heterologous polypeptide using methods known in the art.

It is therefore disclosed a method of improving heterologous polypeptide production in a Trichoderma cell expression system, comprising
a) providing a Trichoderma cell as disclosed above in which one or more regulatory proteins have reduced or eliminated activity, and further comprising a recombinant nucleic acid encoding said heterologous polypeptide,
b) culturing said Trichoderma cell for production of the heterologous polypeptide, wherein the heterologous polypeptide is produced at a higher yield when compared to the heterologous polypeptide produced in a corresponding parental Trichoderma cell in which said one or more regulatory proteins do not have reduced or eliminated activity.

It is further disclosed a method of making a heterologous polypeptide, comprising
a) providing a Trichoderma cell as disclosed above in which one or more regulatory proteins have reduced or eliminated activity;
b) culturing said Trichoderma cell for production and secretion of a heterologous polypeptide in the culture medium; and,
c) recovering and, optionally, purifying the heterologous polypeptide from the culture medium.

For example, the cells may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The secreted polypeptide can be recovered directly from the medium. If the polypeptide is not secreted, it may be obtained from cell lysates.

A heterologous polypeptide of interest produced by a filamentous fungal cell of the present disclosure containing reduced activity in at least one or more of the regulatory proteins of Table 1 may be detected using methods known in the art that are specific for the heterologous polypeptide. These detection methods may include, without limitation, use of specific antibodies, high performance liquid chromatography, capillary chromatography, formation of an enzyme product, disappearance of an enzyme substrate, and SDS-PAGE. For example, an enzyme assay may be used to determine the activity of an enzyme. Procedures for determining enzyme activity are known in the art for many enzymes (see, for example, O. Schomburg and M. Salzmann (eds.), Enzyme Handbook, Springer-Verlag, New York, 1990).

The resulting heterologous polypeptide may be isolated by methods known in the art. For example, a heterologous polypeptide of interest may be isolated from the cultivation medium by conventional procedures including, without limitation, centrifugation, filtration, extraction, spray-drying, evaporation, and precipitation. The isolated heterologous polypeptide may then be further purified by a variety of procedures known in the art including, without limitation, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, for example, Protein Purification, J.-C. Janson and Lars Ryden, editors, VCH Publishers, New York, 1989).

In certain embodiments, a mammalian polypeptide, for example an immunoglobulin or antibody, is produced by the methods of the present disclosure at a level that is at least 2-fold; 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 70-fold, at least 75-fold, at least 80-fold, at least 90-fold, at least 100-fold, or a greater fold higher than the production level of the polypeptide in a corresponding parental filamentous fungal cell without the reduced regulatory protein activity. In other embodiments, the mammalian polypeptide is produced in a full length version at a level higher than the production level of the full-length version of the polypeptide in a corresponding parental filamentous fungal cell.

In certain embodiments, a non-mammalian polypeptide is produced at a level that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 70-fold, at least 75-fold, at least 80-fold, at least 90-fold, at least 100-fold, or a greater fold higher than the production level of the polypeptide in a corresponding parental filamentous fungal cell. In other embodiments, the non-mammalian polypeptide is produced in a full length version at a level higher than the production level of the full-length version of the polypeptide in a corresponding parental filamentous fungal cell.

siRNA Compounds of the Disclosure

In the above methods, the activity of the regulatory proteins of the disclosure may be reduced by contacting the cells, for example the Trichoderma cells, with siRNA compounds directed against one or more of the genes encoding said regulatory proteins.

As used herein the term “siRNA compound” refers to a small interfering RNA, shRNA, siNA, synthetic shRNA; miRNA compounds capable of down-regulating the expression of a gene, also referred as silencing.

The term “down-regulate” or “silencing” as used herein refers to reducing the expression of a gene or the activity of the product of such gene to an extent sufficient to achieve a desired biological or physiological effect. As used herein, the term “silencing” of a target gene means inhibition of the gene expression (transcription or translation) or polypeptide activity of the product of a target gene. “Gene product” as used herein, refers to a product of a gene such as an RNA transcript or a polypeptide. The terms “RNA transcript”, “mRNA polynucleotide sequence”, “mRNA sequence” and “mRNA” are used interchangeably.

siRNA compound may include oligomers with specific sequence complementary to a target gene of interest. As used herein, the term siRNA further refers to a plasmid or expression vector including one or more oligonucleotides encoding one or more siRNAs, operably linked to suitable promoters.

As used herein “oligomer” refers to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The compounds of the present invention encompass molecules comprising deoxyribonucleotides, ribonucleotides, modified deoxyribonucleotides, modified ribonucleotides, nucleotide analogues, modified nucleotide analogues, unconventional and abasic moieties and combinations thereof.

“Nucleotide” is meant to encompass deoxyribonucleotides and ribonucleotides, which may be natural or synthetic and modified or unmodified. Nucleotides include known nucleotide analogues, which are synthetic, naturally occurring, and non-naturally occurring. Examples of such analogs include, without limitation, phosphorothioates, phosphoramidites, methyl phosphonates, chiral-methyl phosphonates, 2′-O-methyl ribonucleotides, and peptide-nucleic acids (PNAs). Modifications include changes to the sugar moiety, the base moiety and or the linkages between ribonucleotides in the oligoribonucleotide. As used herein, the term “ribonucleotide” encompasses natural and synthetic, unmodified and modified ribonucleotides and ribonucleotide analogues which are synthetic, naturally occurring, and non-naturally occurring. Modifications include changes to the sugar moiety, to the base moiety and/or to the linkages between ribonucleotides in the oligonucleotide.

The nucleotides are selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halouracil, 5-halocytosine, 6-azacytosine and 6-az thymine, pseudouracil, deoxypseudouracil, 4-thiouracil, ribo-2-thiouridine, ribo-4-thiouridine, 8-halo adenine, 8-aminoadenine, 8-thioladenine, 8-thiolalkyl adenines, 8-hydroxyl adenine and other 8-substituted adenines, 8-haloguanines, 8-aminoguanine, 8-thiolguanine, 8-thioalkylguanines 8-hydroxy lguanine and other substituted guanines, other aza and deaza adenines, other aza and deaza guanines, 5-methylribouridine, 5-trifluoromethyl uracil, 5-methylribocytosine, and 5-trifluorocytosine. In some embodiments one or more nucleotides in an oligomer is substituted with inosine.

In some embodiments the siRNA compound further comprises at least one modified ribonucleotide selected from the group consisting of a ribonucleotide having a sugar modification, a base modification or an internucleotide linkage modification and may contain DNA, and modified nucleotides such as LNA (locked nucleic acid), ENA (ethylene-bridged nucleic acid), PNA (peptide nucleic acid), arabinoside, phosphonocarboxylate or phosphinocarboxylate nucleotide (PACE nucleotide), or nucleotides with a 6 carbon sugar.

Modified deoxyribonucleotide includes, for example 5′OMe DNA (5-methyl-deoxyriboguanosine-3′-phosphate) which may be useful as a nucleotide in the 5′ terminal position (position number 1); PACE (deoxyriboadenosine 3′ phosphonoacetate, deoxyribocytidine 3′ phosphonoacetate, deoxyriboguanosine 3′ phosphonoacetate, deoxyribothymidine 3′ phosphonoacetate).

Bridged nucleic acids include LNA (2′-0, 4′-C-methylene bridged Nucleic Acid adenosine 3′ monophosphate, 2′-0,4′-C-methylene bridged Nucleic Acid 5-methyl-cytidine 3′ monophosphate, 2′-0,4′-C-methylene bridged Nucleic Acid guanosine 3′ monophosphate, 5-methyl-uridine (or thymidine) 3′ monophosphate); and ENA (2′-0,4′-C-ethylene bridged Nucleic Acid adenosine 3′ monophosphate, 2′-0,4′-C-ethylene bridged Nucleic Acid 5-methyl-cytidine 3′ monophosphate, 2′-0,4′-C-ethylene bridged Nucleic Acid guanosine 3′ monophosphate, 5-methyl-uridine (or thymidine) 3′ monophosphate).

All analogs of, or modifications to, a nucleotide/oligonucleotide are employed with the present invention, provided that said analog or modification does not substantially adversely affect the properties, e.g. function, of the nucleotide/oligonucleotide. Acceptable modifications include modifications of the sugar moiety, modifications of the base moiety, modifications in the internucleotide linkages and combinations thereof.

A sugar modification includes a modification on the 2′ moiety of the sugar residue and encompasses amino, fluoro, alkoxy (e.g. methoxy), alkyl, amino, fluoro, chloro, bromo, CN, CF, imidazole, carboxylate, thioate, CI to CIO lower alkyl, substituted lower alkyl, alkaryl or aralkyl, OCF3, OCN, 0-, S-, or N-alkyl; 0-, S-, or N-alkenyl; SOCH3; S02CH3; ON02; N02, N3; heterocycloalkyl; heterocycloalkaryl; aminoalkylamino; polyalkylamino or substituted silyl, as, among others, described in European patents EP 0 586 520 B1 or EP 0 618 925 B1.

In one embodiment the modified siRNA compound comprises at least one ribonucleotide comprising a 2′ modification on the sugar moiety (“2′ sugar modification”). In certain embodiments the siRNA compound comprises 2′O-alkyl or 2′-fluoro or 2′O-allyl or any other 2′ modification, optionally on alternate positions. Other stabilizing modifications are also possible (e.g. terminal modifications). In some embodiments a preferred 2′O-alkyl is 2′O-methyl (methoxy) sugar modification.

In some embodiments the backbone of the oligonucleotides is modified and comprises phosphate-D-ribose entities but may also contain thiophosphate-D-ribose entities, triester, thioate, 2′-5′ bridged backbone (also may be referred to as 5′-2′), PACE and the like.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me riboU, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments the non-base pairing nucleotide analog is a deoxyribonucleotide. In addition, analogues of polynucleotides may be prepared wherein the structure of one or more nucleotide is fundamentally altered and better suited as therapeutic or experimental reagents. An example of a nucleotide analogue is a peptide nucleic acid (PNA) wherein the deoxyribose (or ribose) phosphate backbone in DNA (or RNA) is replaced with a polyamide backbone which is similar to that found in peptides. PNA analogues have been shown to be resistant to enzymatic degradation and to have enhanced stability in vivo and in vitro. Other modifications include polymer backbones, cyclic backbones, acyclic backbones, thiophosphate-D-ribose backbones, triester backbones, thioate backbones, 2′-5′ bridged backbone, artificial nucleic acids, morpholino nucleic acids, glycol nucleic acid (GNA), threose nucleic acid (TNA), arabinoside, and mirror nucleoside (for example, beta-L-deoxyribonucleoside instead of beta-D-deoxyribonucleoside). Examples of siRNA compounds comprising LNA nucleotides are disclosed in Elmen et al, (NAR 2005, 33(1):439-447).

In some embodiments the compounds of the present invention are synthesized with one or more inverted nucleotides, for example inverted thymidine or inverted adenosine (see, for example, Takei, et al, 2002, JBC 277(26):23800-06).

Other modifications include 3′ terminal modifications also known as capping moieties. Such terminal modifications are selected from a nucleotide, a modified nucleotide, a lipid, a peptide, a sugar and inverted abasic moiety. Such modifications are incorporated, for example at the 3′ terminus of the sense and/or antisense strands.

What is sometimes referred to in the present invention as an “abasic nucleotide” or “abasic nucleotide analog” is more properly referred to as a pseudo-nucleotide or an unconventional moiety. A nucleotide is a monomeric unit of nucleic acid, consisting of a ribose or deoxyribose sugar, a phosphate, and a base (adenine, guanine, thymine, or cytosine in DNA; adenine, guanine, uracil, or cytosine in RNA). A modified nucleotide comprises a modification in one or more of the sugar, phosphate and or base. The abasic pseudo-nucleotide lacks a base, and thus is not strictly a nucleotide.

The term “capping moiety” as used herein includes abasic ribose moiety, abasic deoxyribose moiety, modifications abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′O-Me nucleotide; and nucleotide analogs including 4′,5′-methylene nucleotide; 1-(P-D-erythrofuranosyl)nucleotide; 4′-thionucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Certain preferred capping moieties are abasic ribose or abasic deoxyribose moieties; inverted abasic ribose or abasic deoxyribose moieties; C6-amino-Pi; a mirror nucleotide including L-DNA and L-R A.

A “hydrocarbon moiety or derivative thereof refers to straight chain or branched alkyl moieties and moieties per se or further comprising a functional group including alcohols, phosphodiester, phosphorothioate, phosphonoacetate and also includes amines, carboxylic acids, esters, amides aldehydes. “Hydrocarbon moiety” and “alkyl moiety” are used interchangeably.

“Terminal functional group” includes halogen, alcohol, amine, carboxylic, ester, amide, aldehyde, ketone, ether groups.

The term “unconventional moiety” as used herein refers to abasic ribose moiety, an abasic deoxyribose moiety, a deoxyribonucleotide, a modified deoxyribonucleotide, a mirror nucleotide, a non-base pairing nucleotide analog and a nucleotide joined to an adjacent nucleotide by a 2′-5′ internucleotide phosphate bond; bridged nucleic acids including locked nucleic acids (LNA) and ethylene bridged nucleic acids (ENA).

Abasic deoxyribose moiety includes for example abasic deoxyribose-3′-phosphate; 1,2-dideoxy-D-ribofuranose-3-phosphate; 1,4-anhydro-2-deoxy-D-ribitol-3-phosphate.

Inverted abasic deoxyribose moiety includes inverted deoxyriboabasic; 3′,5′ inverted deoxyabasic 5′-phosphate.

It is herein disclosed in particular a siRNA compounds including a siRNA, for example, one or more siRNAs directed against a gene encoding a regulatory protein selected from the group consisting of ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), prp2 (SEQ ID NO:12), and ptf7 (SEQ ID NO:11). Suitable siRNA sequences are disclosed in the Examples below.

Using public and proprietary algorithms the sequences of potential siRNAs are generated. siRNA molecules according to the above disclosures may thus be synthesized by any of the methods that are well known in the art for synthesis of ribonucleic (or deoxyribonucleic) oligonucleotides. Synthesis is commonly performed in a commercially available synthesizer (available, inter alia, from Applied Biosystems). Oligonucleotide synthesis is described for example in Beaucage and Iyer, Tetrahedron 1992; 48:2223-2311; Beaucage and Iyer, Tetrahedron 1993; 49: 6123-6194 and Caruthers, et. al, Methods Enzymol. 1987; 154: 287-313; the synthesis of thioates is, among others, described in Eckstein, Ann. Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et al, in IRL Press 1989 edited by Oliver R. W. A.; Kap. 7: 183-208.

Other synthetic procedures are known in the art, e.g. the procedures described in Usman et al, 1987, J. Am. Chem. Soc, 109, 7845; Scaringe et al, 1990, NAR., 18, 5433; Wincott et al, 1995, NAR. 23, 2677-2684; and Wincott et al, 1997, Methods Mol. Bio., 74, 59, may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

In some embodiments the oligonucleotides or siRNA compounds of the present disclosure are synthesized separately and joined together post-synthetically, for example, by ligation (Moore et al., 1992, Science 256, 9923; Draper et al., International Patent Publication No. WO 93/23569; Shabarova et al, 1991, NAR 19, 4247; Bellon et al, 1997, Nucleosides & Nucleotides, 16, 951; Bellon et al, 1997, Bioconjugate Chem. 8, 204), or by hybridization following synthesis and/or deprotection.

In a specific embodiment, said siRNA compound of the present disclosure is further directed to a gene encoding a protease, for example as described in the above Table 2. Accordingly, the siRNA compounds include both one or more siRNA directed against one or more regulatory proteins of the disclosure, and one or more siRNA directed against one or more proteases.

More specifically, the siRNA compounds are directed against a gene encoding a regulatory protein of the present disclosure and a protease selected from the group consisting of pep4, pep8, pep9, pep11, slp5, cpa5, cpa2, cpa3, amp3, tpp1, pep12, amp2, mp1, mp2, mp3, mp4, mp5, amp1, sep1, slp2, slp3, slp6, slp7 and slp8. Suitable siRNA sequences are disclosed in the Examples.

Said siRNA compounds may be complexed with a cationic lipid carrier or other suitable carrier to increase the efficiency of siRNA transfection into a fungal cell, for example a Trichoderma cell comprising a recombinant nucleic acid encoding a heterologous polypeptide.

For silencing the gene encoding the regulatory proteins and/or proteases, the siRNA compounds may be introduced into the fungal cells of the present disclosure (i.e. transfected into the cells), by lipofection, electroporation or other appropriate techniques, in an amount sufficient to down-regulate the target genes of interest (regulatory proteins and/or proteases).

Further details of the methods for silencing the genes of interest with the siRNA compounds are given in the Examples below.

### Pharmaceutical Compositions Containing Heterologous Polypeptides Produced by Filamentous Fungal Cells of the Disclosure

In another aspect, the present invention provides a composition, e.g., a pharmaceutical composition, containing one or more heterologous polypeptides of interest, such as mammalian polypeptides, produced by the filamentous fungal cells of the present disclosure having reduced activity of one or more regulatory proteins of the disclosure (see e.g. Table 1) and further containing a recombinant polynucleotide encoding the heterologous polypeptide, formulated together with a pharmaceutically acceptable carrier. Pharmaceutical compositions of the invention also can be administered in combination therapy, i.e., combined with other agents. For example, the combination therapy can include a mammalian polypeptide of interest combined with at least one other therapeutic agent.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., the mammalian polypeptide of interest may be coated in a material to protect the compound from the action of acids and other natural conditions that may inactivate the compound.

The pharmaceutical compositions of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the certain methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of a mammalian polypeptide of interest, in particular where the mammalian polypeptide is an antibody, the dosage ranges from about 0.0001 to 100 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Certain dosage regimens for an antibody may include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

Alternatively a mammalian polypeptide of interest can be administered as a sustained release formulation, in which case less frequent administration is required. Dosage and frequency vary depending on the half-life of the administered substance in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an immunoglobulin of the present disclosure preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A composition of the present disclosure can be administered via one or more routes of administration using one or more of a variety of methods known in the art. As will be appreciated by the skilled artisan, the route and/or mode of administration will vary depending upon the desired results. Certain routes of administration for binding moieties of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a mammalian polypeptide according to the present disclosure can be administered via a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. (see, e.g., Sustained and Controlled Release Drug Delivery Systems, J. R. Robinson, ed., Marcel Dekker, Inc., New York, 1978).

Therapeutic compositions can be administered with medical devices known in the art.

For example, in a certain embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system.

In certain embodiments, the use of mammalian polypeptides according to the present disclosure is for the treatment of any disease that may be treated with therapeutic antibodies.

### Uses of the Filamentous Fungal Cells of the Invention

The invention herein further relates to the use of any of the filamentous fungal cells of the present disclosure, such as Trichoderma fungal cells, that have reduced or no activity of one or more of the regulatory proteins of the disclosure (see e.g. Table 1), and that contain a recombinant polynucleotide encoding a heterologous polypeptide, such as a mammalian polypeptide, for producing said heterologous polypeptide at high level, or for improving heterologous polypeptide stability and for making a heterologous polypeptide. Methods of measuring protein production and for making a heterologous polypeptide are well known, and include, without limitation, all the methods and techniques described in the present disclosure.

Accordingly, certain embodiments of the present disclosure relate to methods of improving heterologous polypeptide production, by: a) providing a filamentous fungal cell of the present disclosure having reduced or no activity of one or more of the regulatory proteins selected from ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), prp2 (SEQ ID NO:12), and ptf7 (SEQ ID NO:11), and where the cell further contains a recombinant polynucleotide encoding a heterologous polypeptide; and b) culturing the cell such that the heterologous polypeptide is produced, where the heterologous polypeptide is produced at a higher yield and/or increased stability when compared to the yield of the same heterologous polypeptide produced in a host cell in which said one or more regulatory proteins do not have reduced or no activity, for example, in a host cell not containing any mutations of the genes encoding the regulatory proteins, that reduce or eliminate the expression of said regulatory proteins.

Other embodiments of the present disclosure relate to methods of improving mammalian polypeptide stability, by: a) providing a Trichoderma fungal cell of the present disclosure having reduced or no activity of one or more of the regulatory proteins selected from ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), prp2 (SEQ ID NO:12), and ptf7 (SEQ ID NO:11), where the cell further contains a recombinant polynucleotide encoding a mammalian polypeptide; and b) culturing the cell such that the mammalian polypeptide is expressed, where the mammalian polypeptide has increased stability compared to a host cell not containing the mutations of the genes encoding the proteases.

The filamentous fungal cell or Trichoderma fungal cell may be any cell described in the section entitled “Filamentous Fungal Cells of the Invention”.

Other embodiments of the present disclosure relate to methods of making a heterologous polypeptide, by: a) providing a filamentous fungal cell, for example a Trichoderma cell of the present disclosure having reduced or no activity in said one or more regulatory protein selected from the group consisting of one or more of the regulatory proteins selected from ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), prp2 (SEQ ID NO:12), and ptf7 (SEQ ID NO:11), where the cell further contains a recombinant polynucleotide encoding a heterologous polypeptide; b) culturing the host cell such that the heterologous polypeptide is produced and secreted in the culture medium; and c) recovering and, optionally, purifying the heterologous polypeptide.

Methods of culturing filamentous fungal and Trichoderma fungal cells and purifying polypeptides are well known in the art, and include, without limitation, all the methods and techniques described in the present disclosure.

In certain embodiments, the filamentous fungal cell or Trichoderma fungal cell is cultured at a pH range selected from pH 3.5 to 7; pH 3.5 to 6.5; pH 4 to 6; pH 4.3 to 5.7; pH 4.4 to 5.6; and pH 4.5 to 5.5. In certain embodiments, to produce an antibody the filamentous fungal cell or Trichoderma fungal cell is cultured at a pH range selected from 4.7 to 6.5; pH 4.8 to 6.0; pH 4.9 to 5.9; and pH 5.0 to 5.8.

In some embodiments, the heterologous polypeptide is a mammalian polypeptide. In other embodiments, the heterologous polypeptide is a non-mammalian polypeptide.

In certain embodiments, the mammalian polypeptide is selected from an immunoglobulin, immunoglobulin heavy chain, an immunoglobulin light chain, a monoclonal antibody, a hybrid antibody, an F(ab′)2 antibody fragment, an F(ab) antibody fragment, an Fv molecule, a single-chain Fv antibody, a dimeric antibody fragment, a trimeric antibody fragment, a functional antibody fragment, a single domain antibody, multimeric single domain antibodies, an immunoadhesin, insulin-like growth factor 1, a growth hormone, insulin, and erythropoietin. In other embodiments, the mammalian protein is an immunoglobulin or insulin-like growth factor 1. In yet other embodiments, the mammalian protein is an antibody. In further embodiments, the yield of the mammalian polypeptide is at least 0.5, at least 1, at least 2, at least 3, at least 4, or at least 5 grams per liter. In certain embodiments, the mammalian polypeptide is an antibody, optionally, IgG1, IgG2, IgG3, or IgG4. In further embodiments, the yield of the antibody is at least 0.5, at least 1, at least 2, at least 3, at least 4, or at least 5 grams per liter. In still other embodiments, the mammalian polypeptide is a growth factor or a cytokine. In further embodiments, the yield of the growth factor or cytokine is at least 0.1, at least 0.2, at least 0.3, at least 0.4, at least 0.5, at least 1, at least 1.5, at least 2, at least 3, at least 4, or at least 5 grams per liter. In further embodiments, the mammalian polypeptide is an antibody, and the antibody contains at least 70%, at least 80%, at least 90%, at least 95%, or at least 98% of a natural antibody C-terminus and N-terminus without additional amino acid residues. In other embodiments, the mammalian polypeptide is an antibody, and the antibody contains at least 70%, at least 80%, at least 90%, at least 95%, or at least 98% of a natural antibody C-terminus and N-terminus that do not lack any C-terminal or N-terminal amino acid residues

In certain embodiments where the mammalian polypeptide is purified from cell culture, the culture containing the mammalian polypeptide contains polypeptide fragments that make up a mass percentage that is less than 50%, less than 40%, less than 30%, less than 20%, or less than 10% of the mass of the produced polypeptides. In certain preferred embodiments, the mammalian polypeptide is an antibody, and the polypeptide fragments are heavy chain fragments and/or light chain fragments. In other embodiments, where the mammalian polypeptide is an antibody and the antibody purified from cell culture, the culture containing the antibody contains free heavy chains and/or free light chains that make up a mass percentage that is less than 50%, less than 40%, less than 30%, less than 20%, or less than 10% of the mass of the produced antibody. Methods of determining the mass percentage of polypeptide fragments are well known in the art and include, measuring signal intensity from an SDS-gel.

In further embodiments, the non-mammalian polypeptide is selected from an aminopeptidase, amylase, carbohydrase, carboxypeptidase, catalase, cellulose, chitinase, cutinase, deoxyribonuclease, esterase, alpha-galactosidase, beta-galactosidase, glucoamylase, alpha-glucosidase, beta-glucosidase, invertase, laccase, lipase, mutanase, oxidase, pectinolytic enzyme, peroxidase, phospholipase, phytase, polyphenoloxidase, proteolytic enzyme, ribonuclease, transglutaminase, and xylanase.

In certain embodiments of any of the disclosed methods, the method includes the further step of providing one or more, two or more, three or more, four or more, or five or more protease inhibitors. In certain embodiments, the protease inhibitors are peptides that are co-expressed with the mammalian polypeptide. In other embodiments, the inhibitors are siRNA compounds which inhibit at least two, at least three, or at least four proteases from a protease family selected from aspartic proteases, trypsin-like serine proteases, subtilisin proteases, and glutamic proteases.

In certain embodiments of any of the disclosed methods, the filamentous fungal cell or Trichoderma fungal cell also contains a carrier protein. As used herein, a “carrier protein” is portion of a protein that is endogenous to and highly secreted by a filamentous fungal cell or Trichoderma fungal cell. Suitable carrier proteins include, without limitation, those of T. reesei mannanase I (Man5A, or MANI), T. reesei cellobiohydrolase II (Cel6A, or CBHII) (see, e.g., Paloheimo et al Appl. Environ. Microbiol. 2003 December; 69(12): 7073-7082) or T. reesei cellobiohydrolase I (CBHI). In some embodiments, the carrier protein is CBH1. In other embodiments, the carrier protein is a truncated T. reesei CBH1 protein that includes the CBH1 core region and part of the CBH1 linker region. In some embodiments, a carrier such as a cellobiohydrolase or its fragment is fused to an antibody light chain and/or an antibody heavy chain. In some embodiments, a carrier such as a cellobiohydrolase or its fragment is fused to insulin-like growth factor 1, growth hormone, insulin, interferon alpha 2b, fibroblast growth factor 21, or human serum albumin. In some embodiments, a carrier-antibody fusion polypeptide comprises a Kex2 cleavage site. In certain embodiments, Kex2, or other carrier cleaving enzyme, is endogenous to a filamentous fungal cell. In certain embodiments, carrier cleaving protease is heterologous to the filamentous fungal cell, for example, another Kex2 protein derived from yeast or a TEV protease. In certain embodiments, carrier cleaving enzyme is overexpressed.

It is to be understood that, while the invention has been described in conjunction with the certain specific embodiments thereof, the foregoing description is intended to illustrate and not limit the scope of the invention. Other aspects, advantages, and modifications within the scope of the invention will be apparent to those skilled in the art to which the invention pertains.

The invention having been described, the following examples are offered to illustrate the subject invention by way of illustration, not by way of limitation.

### EXAMPLES

### Example 1. Protease Induction Studies

To search for transcriptional regulators of proteases in Trichoderma reesei, an induction experiments were performed to upregulate protease expression and activity. Addition of 10 g/l casamino acids or 4 g/l peptone to Trichoderma minimal medium (TrMM) containing lactose and 20 g/l spent grain extract medium increased the total protease activity in the culture supernatants of the M180 Trichoderma reesei strain producing a monoclonal antibody MAB01 (no regulatory proteins or proteases are deleted in the M180 strain).

The M180 strain was grown in 2 liter flasks with 250 ml of TrMM, 40 g/l lactose, 20 g/l spent grain extract, supplemented with 100 mM PIPPS pH 5.5. The culture was grown up so that the dry weight was 0.8 g/l. The whole batch of growing mycelia was pooled and equally distributed resulting to 250 ml in four replicate 2 liter flasks per treatment. Protease induction was started by addition of casamino acids or peptone. A mock induction was done with buffer for the uninduced cultures. Casamino acids were added to give a final concentration of 10 g/l and peptone was added to give a final concentration of 4 g/l.

The flasks were sampled after adding the inducers so the zero time point is several minutes after induction began. In total 40 ml samples were taken at 0, 8 hours, 20 hours and 48 hours post induction, the mycelium was isolated, and frozen in liquid nitrogen subsequent RNA isolation (altogether for each time point there were 4 samples taken from 4 independent flasks).

Custom-made microarray slides from RocheNimbleGen were used for transcriptional profiling. Sample preparation, hybridization onto microarray slides and collection of raw data was carried out according to the manufacturer's protocols. The microarray data were analyzed using the R package Oligo for preprocessing of the data and the package Limma for identifying differentially expressed genes. In the analysis of the differentially expressed genes, the signals in the samples of the induced cultures were compared to the ones in the uninduced control cultures at the corresponding time point. Four biological replicates of each condition and time point were analyzed. In addition, the expression array datasets were clustered using the R package Mfuzz. Co-expressed genomic clusters were determined by enrichment of Mfuzz cluster members in the same genomic regions.

The expression data was clustered into 100 groups to find out what genes are co-regulated upon the different treatment. The samples clustered as expected. The early time points 0 and 8 hours are clearly separated from the 20 and 48 hour time points. At 8 hours there are distinguishable differences between the induced cultures and the uninduced control. At 20 hours the induced cultures are most different compared to the control cultures. After 48 hours the peptone induced cultures have become more like the control cultures. This would indicate that the peptone has been used up and the cultures are equilibrating to become more like the untreated control. In the further analysis we focused on time points where both treatments would give upregulation of protease genes and regulatory factors. There was some activity at 8 hours, but the majority of information was gathered from the 20 hour time point.

At 20 hours there were 956 genes that were upregulated at least 1.4-fold in both treatments using a p-value=0.01. At 8 hours there were 375 genes similarly upregulated. From these, 26 protease genes were upregulated in total, while 20 were upregulated in both treatments at 20 hours. Only 6 proteases were upregulated in total, with 3 of the same upregulated in both treatments at 8 hours. 41 transcription factors/regulating proteins were found to be upregulated at least 1.4-fold in both treatments at 20 hours, while there were only 14 similar genes at 8 hours.

To narrow down the selection, the transcription factors were located on the scaffold to see if they were physically near to any protease genes. 7 transcription factor/regulators were found to be near at least 2 protease genes. Combining this analysis with the clustering data revealed that 3 of the 7 transcription factors cluster with 3 or more protease genes. Table 3 below summarizes the candidate transcriptional regulators. The most interesting genes are the transcription factors tre3449 and tre108940, and the regulatory protein tre122069. The protease genes found in the same expression clusters are listed in Table 4. For example, the transcription factor tre3449 is next to tre121306 aspartic protease on the gene scaffold, which could suggest a relationship between the regulator and the gene.

TABLE 3
Candidate transcription factors/regulatory proteins that are involved in upregulating
protease expression. The time point is indicated along with the number of protease genes that the
transcription factor/regulatory protein clusters with and the number of protease genes nearby the
gene locus.

| | | Protease | | | |
|---|---|---|---|---|---|
| Time | Proteases | genes | | | SEQ ID |
| point | in cluster | nearby | Gene # | Gene description | NO: |
| 20 h | 2 | 2 | tre59740 | Fungal transcriptional regulatory protein, N- | |
| | | | | terminal | |
| 20 h | 3 | 2 | tre3449 | Fungal transcriptional regulatory protein, N- | |
| | | | | terminal | |
| 20 h | 1 | 2 | tre106706 | Fungal transcriptional regulatory protein, N- | |
| | | | | terminal | |
| 20 h | 7 | 2 | tre122069 | BTB/POZ | |
| 20 h | 4 | 2 | tre108940 | Fungal transcriptional regulatory protein, N- | |
| | | | | terminal | |
| 20 h | 1 | 2 | tre103158 | Fungal specific transcription factor | |
| 20 h | 2 | 1 | tre103275 | Zn-finger, C2H2 type | |
| 20 h | 1 | 1 | tre105269 | Fungal transcriptional regulatory protein, N- | |
| | | | | terminal | |
| 20 h | 2 | 1 | tre76505 | Zn-finger, C2H2 type | |
| 8 h | 2 | 2 | tre121130 | Fungal transcriptional regulatory protein, N- | |
| | | | | terminal | |
| 8 h | 3 | 1 | tre106259 | Fungal specific transcription factor | |
| 8 h | 3 | 1 | tre102947 | BTB/POZ | |

The dataset was reduced down to only regulatory proteins, and protease genes and clustered again to find out what genes were co-regulated within a smaller set of data. There were two clustering sets of genes including these three factors. The tre3449 and tre122069 strongly clustered together along with several protease genes and one transcription factor tre76505. The tre108940 gene clusters well with several of the most major proteases and with another transcription factor tre106706. These genes appear to be co-regulated and are listed in Table 5.

There were at least 17 protease genes that were upregulated at least 2-fold upon induction with casamino acids or peptone (Table 6). The majority of the induction occurred at 20 hours. At 48 hours, there was still induction from the casamino acids, but in the peptone treated cultures the protease expression went back to normal. The most highly upregulated proteases with high transcription levels included tsp1, slp1, pep1, pep2, pep5, pep10, and pep11.

TABLE 6
Protease genes that were upregulated at least 2-fold after induction with 1% casamino
acids or 0.4% peptone at different time points in the induction study.

| | | p = 0.01, >2 fold | | | | | | | | |
|---|---|---|---|---|---|---|---|---|---|---|
| | | 1% casamino acid | | | 0.4% peptone | | | Control signal intensity | | |
| | | 8 hr | 20 hr | 48 hr | 8 hr | 20 hr | 48 hr | 8 hr | 20 hr | 48 hr |
| tpp1 | tre0082623 | 0 | 1 | 0 | 0 | 1 | 0 | 14.3 | 10.8 | 8.8 |
| tsp1 | tre0073897 | 0 | 0 | 1 | 0 | 1 | 0 | 9.2 | 8.2 | 9.0 |
| sep1 | tre0124051 | −1 | 1 | 0 | −1 | 1 | 0 | 12.6 | 10.0 | 7.8 |
| slp1 | tre0051365 | 0 | 1 | 0 | 0 | 1 | 0 | 13.8 | 12.8 | 12.4 |
| pep1 | tre0074156 | −1 | 1 | 1 | −1 | 1 | 0 | 13.2 | 12.3 | 8.4 |
| pep10 | tre0078639 | 0 | 0 | 1 | 0 | 0 | 0 | 11.1 | 12.7 | 9.6 |
| pep11 | tre0121306 | 0 | 0 | 1 | 0 | 0 | 0 | 14.0 | 13.0 | 10.7 |
| pep2 | tre0053961 | 1 | 1 | 0 | 1 | 1 | 0 | 10.3 | 8.9 | 10.5 |
| slp5 | tre0064719 | 0 | 1 | 0 | 0 | 0 | 0 | 11.0 | 9.9 | 10.0 |
| pep5 | tre0081004 | 0 | 0 | 1 | 0 | 0 | 0 | 8.5 | 8.0 | 11.8 |
| slp8 | tre0058698 | 0 | 1 | 0 | 0 | 1 | 0 | 9.0 | 7.3 | 6.8 |
| pep17 | tre0111818 | 1 | 1 | 0 | 1 | 1 | 0 | 7.6 | 5.5 | 6.5 |
| slp3 | tre0123234 | 0 | 1 | 0 | 0 | 1 | 0 | 6.5 | 4.7 | 5.3 |
| amp6 | tre0081087 | 0 | 0 | 0 | −1 | 1 | 0 | 8.0 | 7.9 | 8.5 |
| amp8 | tre0105279 | 0 | 1 | 0 | 0 | 1 | 0 | 5.3 | 3.6 | 3.4 |
| slp6 | tre0121495 | −1 | 0 | 0 | −1 | 1 | 0 | 6.8 | 4.4 | 4.8 |
| slp9 | tre0060791 | 0 | 0 | 1 | 0 | 0 | 0 | 5.2 | 4.2 | 4.6 |

1 = upregulated and −1 = downregulated.
0 = no change.

### Example 2 Transient siRNA Silencing of Genes

The five most interesting regulatory genes were downregulated via siRNA based gene silencing treatments and the effect on protease genes was monitored with qPCR. The siRNA transformation was done using protoplasts from the M180 strain. This strain does not have any protease deletions and carries expression constructs for production of MAB01 antibody (MAB01 antibody is described in WO/2013/102674). The siRNAs were complexed with a cationic lipid carrier (GeneSilencer). The mixture was 5 μl of carrier per 0.3 nmoles of siRNA combined in a complexing buffer provided by the kit and incubated for 20 minutes before adding to the protoplasts. After adding the protoplasts to the siRNA/carrier mixture the concentration of each siRNA was 200 pmol/ml. Three separate siRNAs were used together for each gene to ensure a successful knockdown (Table 7).

The protoplasts (3×106) were added to 200 μl of media (TrMM 4 g/l lactose, 1 g/l yeast extract, 1.2M sorbitol) and then the whole siRNA/lipid carrier mixture was mixed with the protoplasts. These were incubated together at room temperature for 15 minutes before adding 2800 μl of media and adding to a 24 well plate. These were grown in 28° C. on a shaker set for 80 rpm. Each day 1 ml was collected and the mycelium/protoplast mixture was spun down for 5 minutes at 13 k. The culture was sampled for 3 days. The supernatant was removed and the cell pellets were flash frozen and stored at −80° C. The RNA was extracting using the RNeasy mini kit (Qiagen). The total RNA was made into cDNA using the transcriptor high fidelity cDNA synthesis kit (Roche). The cDNA were amplified with qPCR using gene specific primers and gpdl primers for normalization. The qPCR was done with SYBR green I and a Roche lightcycler 480 machine. Primers are listed in Table 8.

TABLE 7
siRNAs targeting regulatory proteins under study.

| siRNA name | siRNA sequence |
|---|---|
| TRIRE3449 | GCCAUUUCGGCUAGCAUCATT |
| TRIRE3449_2 | GUAUUCAUCGGUAUCCUUUTT |
| TRIRE3449_3 | GGAUAAGUCACUUUCUGAATT |
| TRIRE122069 | GUCUGACUCGGACAUUAAGTT |
| TRIRE122069_2 | GCAUGGUGGACUUCUUCUATT |
| TRIRE122069_3 | CUCUCUCACAGAUCAAGUUTT |
| TRIRE105269 | CUCAUGUUGGCUGACGGAATT |
| TRIRE105269_2 | GCAAUCUCGCCGGCUCAAUTT |
| TRIRE105269_3 | GCAAGAUGCUUCCGUCACUTT |
| TRIRE76505 | GGCAAGAAGUUCUCUCGCATT |
| TRIRE76505_2 | CUGUGUAUCUCCAGUCCCATT |
| TRIRE76505_3 | CCCUUUGAGUGCAACGAGUTT |
| TRIRE59740 | GGUGCACGUUGCUGCCAAUTT |
| TRIRE59740_2 | CACGUUGCUGCCAAUUCAUTT |
| TRIRE59740_3 | GGUUGGACGUGGUCAUGUUTT |

TABLE 8
List of qpcr primer sequences

| | Gene | |
|---|---|---|
| | abbrevi- | |
| Primer name | ation | Primer sequence |
| tre122069 fwd | prp1 | CTCAATCGCACGCTATTAGTC |
| qpcr | | |
| tre122069 rev | prp1 | AACTCGCAGTCCTTCATCTC |
| qpcr | | |
| tre3449 fwd qpcr | ptf1 | TTTGATGAACGCCAATACCT |
| tre3449 rev qpcr | ptf1 | GATGTATGACCTGGCTAACC |
| tre105269 fwd | ptf2 | ATGTTGGGTCGTTTGTTCAC |
| qpcr | | |
| tre105269 rev | ptf2 | TGATCCGTATGTCTTGTTCC |
| qpcr | | |
| tre59740 fwd qpcr | ptf3 | ACCTTCTCCAGCAGTTTACC |
| tre59740 rev qpcr | ptf3 | CAGGGTGGGAATATAACGTC |
| tre76505 fwd qpcr | ptf4 | CTGTGTATCTCCAGTCCCAG |
| tre76505 rev qpcr | ptf4 | CAGAAGTCAACATCGTGCTC |
| tre77579 fwd qpcr | pep4 | GAATGCCCAATACTTCTCTG |
| tre77579 rev qpcr | pep4 | GCTACCTGATCCGTAGTG |
| tre122076 fwd | pep8 | TCTGTCCAACCAAAGAGTCC |
| qpcr | | |
| tre122076 rev | pep8 | AAAGGTATCGGAGTCATCAGG |
| qpcr | | |
| tre79807 fwd qpcr | peP9 | TGAAGGAGCTTGAAGAACAC |
| tre79807 rev qpcr | peP9 | CTTTAGGATGGAGAACTGATTGTC |
| tre121306 fwd | pep11 | CAGTACAACCATTCCACCAC |
| qpcr | | |
| tre121306 rev | pep11 | TTGCTGATTGATACGCAGAG |
| qpcr | | |
| tre64719 fwd qpcr | slp5 | GTAACGAGAACCAAGACACTG |
| tre64719 rev qpcr | slp5 | CAGGAGCATAGATGTCAACAC |
| tre22210 fwd qpcr | cpa2 | TAGACGGATTCCTGTATACCC |
| tre22210 rev qpcr | cpa2 | GGGACATTCCACATATAGTTCCA |
| tre22459 fwd qpcr | cpa3 | GAACTCAGTCCTCATCCACC |
| tre22459 rev qpcr | cpa3 | ATGTTGCCGTTGAAGAAGAC |
| tre23475 fwd qpcr | amp3 | AAGACTGGAGGAGTGATTTGG |
| tre23475 rev qpcr | amp3 | GTCTCCCTCAAAGTAGTAGCC |
| gpd1 fwd qpcr | gpd1 | TCCATTCGTGTCCCTACC |
| gpd1 rev qpcr | gpd1 | AGATACCAGCCTCAATGTC |

We tested siRNA silencing of tre3449, tre122069, tre105269, tre76505, and tre59740. The tre76505 expression was reduced 2.2-fold, tre105269 was reduced 2.4-fold, and tre59740 was reduced by 4.7-fold, tre122069 reduced 1.3-fold, and tre3449 reduced by 1.7-fold. These experiments proved that the silencing siRNAs chosen indeed silence their target gene. The tre59740, tre3449, and tre122069 were investigated in more detail to evaluate if proteases gene expression was affected by silencing these genes.

The siRNAs treatments were done separately for tre3449 and tre122069 and in combination. When tre3449 siRNAs were transformed into the protoplasts, a clear downregulation of the tre3449 gene (ptf1, protease transcription factor 1) could be seen compared to a lipid carrier only treatment (Table 9). The siRNA treatment was most effective on day 3 where it reduced the expression of ptf1 by 1.7-fold. Likewise the best day for knockdown of tre122069 (prp1, protease regulatory protein 1) was day 3, where the expression of prp1 was reduced 1.3-fold.

Very interestingly, combined treatment with ptf1 and prp1 siRNAs increased the effectiveness of the knockdown. In the combined treatment, day 2 showed the most effective knockdown of both genes. The ptf1 expression was reduced 4.3-fold and prp1 was reduced 4.1-fold (Table 10). When analysing the single treatments for cross regulation of expression it can be observed that knockdown of ptf1 also effected the expression of prp1. On day 2, ptf1 siRNA resulted in 1.8-fold downregulation of prp1 and significant reduction on day 1 and 3, as well. The effect of prp1 siRNA treatment was less pronounced and it resulted in a small 1.4-fold change in the ptf1 expression on day 3 only (Table 10). The ptf1 may be involved in regulating prp1 and could possibly use prp1 as a binding partner.

The goal of these knock down studies was to determine whether reduction in the expression of ptf1 and/or prp1 would affect protease gene expression. In this experiment, the pep11 protease (tre121306) was used as a representative protease to monitor its expression as a result of the siRNA treatments on all days of the experiment. The pep11 protease gene sits right next to ptf1 on the chromosome and was found to co-regulate with ptf1 in the protease induction experiments.

When ptf1 was knocked down by siRNA the expression of pep11 was reduced 1.3- and 1.5-fold on day 1 and 2 (Table 11). The knockdown of prp1 did not affect the expression of pep11 until day 3. However, the knockdown effect of prp1 on itself was not seen until day 3. On day 3 the prp1 siRNA treatment reduced pep11 expression 1.4-fold. However, the combined treatment with both ptf1 and prp1 siRNAs boosted the ability to down regulate pep11 up to 3-fold on day 2. The two regulator proteins appear to be involved together in the ability to reduce protease expression.

The effect of silencing ptf1 and ptf1/prp1 on protease expression was expanded to a larger set of protease genes. These were protease genes that were not already deleted in the strain. The day 1 samples were analyzed in a different qPCR run to see how pep4, pep8, pep9, pep11, slp5, cpa2, cpa3, and amp3 protease were affected after knockdown. The treatment with ptf1 siRNA did not affect the expression of pep4 or slp5, but all of the other proteases were expressed less after treatment. The combination of ptf1/prp1 siRNAs increased the magnitude of the effect, but did not affect the overall result (Table 11). These proteases were also chosen because they appeared to be co-regulated in some way with ptf1 and prp1. The aspartic proteases pep8, pep9, and pep11 could be membrane associated proteases, while the pep4 protease appears to be partly secreted. This limited set of data could suggest that there may be some regulation of membrane bound aspartic proteases. The same set of proteases were investigated and it was found that tre59740 (ptf3) reduced the expression of pep11 by 2.3-fold, slp5 by 1.9-fold, and cpa3 by 4.5-fold.

TABLE 9
qPCR analysis of the expression changes after combined treatment with
tre3449 and tre122069 siRNAs compared to the control. The combined
treatment resulted in a more potent downregulation, with the peak
effectiveness being on day 2.
FOLD knockdown of mRNA expression

| | tre3449 | tre122069 | | |
|---|---|---|---|---|
| | treatment | treatment | | combined treatment |
| | tre3449 | tre122069 | combined treatment | tre122069 |
| | expression | expression | tre3449 expression | expression |
| day 1 | 1.5 | 1.0 | 2.4 | 3.3 |
| day 2 | 1.7 | 1.0 | 4.3 | 4.1 |
| day 3 | 1.7 | 1.3 | 1.8 | 2.4 |

TABLE 10
qPCR analysis of the expression changes after treatment with tre3449
or tre122069 siRNA compared to the control. Treatment with tre3449
siRNA affects tre122069 expression and vice versa suggesting
some form on cross regulation.
FOLD knockdown of mRNA expression

| | tre3449 treatment | tre122069 treatment |
|---|---|---|
| | tre122069 expression | tre3449 expression |
| day 1 | 1.5 | 1.0 |
| day 2 | 1.8 | 0.9 |
| day 3 | 1.7 | 1.4 |

TABLE 11
qPCR analysis of the expression changes after treatment with tre3449,
tre122069, and tre3449/tre122069 siRNA compared to the control. All
these treatments downregulated pep11 expression, but the most potent
reduction in expression was observed with the combined treatment.
FOLD knockdown of mRNA expression

| | tre3449 treatment | | |
|---|---|---|---|
| | tre121306 | tre122069 treatment | combined treatment |
| | expression | tre121306 expression | tre121306 expression |
| day 1 | 1.3 | 1.2 | 2.2 |
| day 2 | 1.5 | 1.0 | 3.1 |
| day 3 | 1.3 | 1.4 | 1.4 |

TABLE 12
qPCR analysis of the expression changes after treatment with tre3449,
tre3449/tre122069, or tre59740 siRNA compared to the control.
These tre3449/tre122069 treatments downregulated the expression
of pep8, pep9, pep11, cpa2, cpa3, and amp3, but not pep4 and slp5.
The combined treatment was more effective at reducing the protease
expression.
The tre59740 siRNA affected pep11, slp5, and cpa3.

| | tre3449 siRNA | tre3449/122069 | tre59740 siRNA |
|---|---|---|---|
| | treatment | siRNA treatment | treatment |
| expression: | fold knockdown | fold knockdown | fold knockdown |
| tre3449 | 1.5 | 2.4 | 1.0 |
| tre122069 | 1.5 | 2.4 | 1.0 |
| pep4 | 1.2 | 1.0 | 1.0 |
| pep8 | 1.4 | 2.4 | 1.0 |
| pep9 | 2.0 | 2.5 | 1.0 |
| pep11 | 1.5 | 2.7 | 2.3 |
| slp5 | 1.0 | 1.1 | 1.9 |
| cpa2 | 1.8 | 3.3 | 1.0 |
| cpa3 | 1.4 | 2.8 | 4.5 |
| amp3 | 1.6 | 3.0 | 1.0 |

### Example 3. Creation of Interferon Expression Strains with Regulatory Protein Deletions

The Trichoderma reesei interferon production strain M577 was used to investigate how deletion of protease regulators affected interferon expression and protease activity. The M577 strain was generated as described in WO/2013/102674. The pyr4-version of M577, M788, was described in WO/2015/004241.

### Generation of Prp1 Deletion Plasmid

The deletion plasmid pTTv273 for the transcription factor prp1 (tre122069) was constructed essentially as described for pep1 deletion plasmid pTTv41 in WO/2013/102674, except that the marker used for selection was pyr4-hgh from pTTv194.

939 bp of 5′ flanking region and 943 bp of 3′ flanking region were selected as the basis of the prp1 deletion plasmid pTTv273. These fragments were amplified by PCR using the primers listed in Table 13. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Apep4-pyr-hgh) with NotI digestion. To enable removal of the marker cassette, NotI restriction sites were introduced on both sides of the cassette. Vector backbone was EcoRI/XhoI digested pRS426 as in WO/2013/102674. The plasmid pTTv273 was constructed with the 5′ flank, 3′ flank, pyr4-hgh marker, and vector backbone using the yeast homologous recombination method described in WO/2013/102674. This deletion plasmid for prp1 (pTTv273, Table 13) results in a deletion in the prp1 locus and covers the complete coding sequence of PRP1.

TABLE 13
Primers for generating prp1 deletion plasmids.
Deletion plasmid pTTv273 (Δprp1-pyr4-hgh), vector
backbone pRS426

| Primer | Sequence |
|---|---|
| T1035_122069_ | GTAACGCCAGGGTTTTCCCAGTCACGACGGTTT |
| 5flkfw_vector | AAACATGGGGATTGGAGAGTGATG |
| T1036_122069_ | GCGCTGGCAACGAGAGCAGAGCAGCAGTAGTCG |
| 5flkrev_pyr4Prom | ATGCTAGGCGGCCGCGAAGCAACCGAGGTGAAA |
| | AG |
| T1037_122069_ | CAACCAGCCGCAGCCTCAGCCTCTCTCAGCCTC |
| 3flkfw_pyr4loop | ATCAGCCGCGGCCGCACGATGCAGGTTTGGTTT |
| | TC |
| T1038_122069_ | GCGGATAACAATTTCACACAGGAAACAGCGTTT |
| 3flkrev_vector | AAACCACCGGAATTGTCTGACCTT |

### Generation of Prp1 Deletion Strain

To isolate the deletion cassette, plasmid pTTv273 was digested with Pinel and the correct fragment purified from an agarose gel using a QIAquick Gel Extraction Kit (Qiagen). Approximately 5 μg of the deletion cassette was used to transform the M577 strain. Preparation of protoplasts and transformation were carried out essentially as described in WO/2013/102674 using hygromycin selection.

Transformants were picked as first streaks. Growing streaks were screened by PCR (using the primers listed in Table 14) for correct integration. Clones giving the expected signals were purified to single cell clones and rescreened by PCR using the primers listed in Table 12. The final selected strain was called M675 (M577 with Δprp1).

TABLE 14
Primers for screening prp1 integration and strain
purity.
For screening integration of pTTv273

| Primer | Sequence |
|---|---|
| T1039_122069_screen_5flk_fwd | CACCAACTGGCAAGTCTCAA |
| T1014_screen_5flk_pyr_rev | GGAGAATTTCGTGCGATCC |
| T1015_screen_3flk_hygro_fwd | GCATGGTTGCCTAGTGAATG |
| T1040_122069_screen_3flk_rev | TGATCTCGCGAAGAACCTTT |

For screening deletion of prp1 ORF

| Primer | Sequence |
|---|---|
| T1041_122069_orf_fwd | GCCTCGGGGAGATATGAGAT |
| T1042_122069_orf_rev | AAAATTCCAAGGCGACACAC |

Generation of ptf1 Deletion Plasmid

The deletion plasmid pTTv372 for the transcription factor ptf1 (tre3449) was constructed essentially as described for pep1 deletion plasmid pTTv41 in WO/2013/102674, except that the marker used for selection was pyr4-hgh from pTTv194.

940 bp of 5′ flanking region and 963 bp of 3′ flanking region were selected as the basis of the ptf1 deletion plasmid pTTv372. These fragments were amplified by PCR using the primers listed in Table 13. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Apep4-pyr-hgh) with NotI digestion. To enable removal of the marker cassette, NotI restriction sites were introduced on both sides of the cassette. Vector backbone was EcoRI/XhoI digested pRS426 as in WO/2013/102674. The plasmid pTTv372 was constructed with the 5′ flank, 3′ flank, pyr4-hgh marker, and vector backbone using the yeast homologous recombination method described in WO/2013/102674. This deletion plasmid for ptf1 (pTTv372, Table 15) results in a deletion in the ptf1 locus and covers the complete coding sequence of PTF1.

TABLE 15
Primers for generating ptf1 deletion plasmids.
Deletion plasmid pTTv372 (Δptf1-pyr4-hgh), vector
backbone pRS426

| Primer | Sequence |
|---|---|
| T1043_3449_ | GTAACGCCAGGGTTTTCCCAGTCACGACGGTTT |
| 5flkfw_vector | AAACCGGAGCTGGGTAGAAGTGTC |
| T1044_3449_ | GCGCTGGCAACGAGAGCAGAGCAGCAGTAGTCG |
| 5flkrev_pyr4Prom | ATGCTAGGCGGCCGCTGATGGAAGAGAGGGTTG |
| | AGA |
| T1308_3449_ | CAACCAGCCGCAGCCTCAGCCTCTCTCAGCCTC |
| 3flkfw_pyr4loop | ATCAGCCGCGGCCGCCAATCCCGAGACTCAACT |
| | CC |
| T1309_3449_ | GCGGATAACAATTTCACACAGGAAACAGCGTTT |
| 3flkrev_vector | AAACTCAGAACATGATCCACTCGAC |

Generation of ptf1 Deletion Strains

To isolate the deletion cassette, plasmid pTTv372 was digested with Pinel and the correct fragment purified from an agarose gel using a QIAquick Gel Extraction Kit (Qiagen). Approximately 5 μg of the pTTv372 deletion cassette was used to transform the M788 pyr4-strain and the M755 pyr4-strain of M675 (prp1 deletion). Preparation of protoplasts and transformation were carried out essentially as described in WO/2013/102674 using hygromycin selection.

Transformants were picked as first streaks. Growing streaks were screened by PCR (using the primers listed in Table 16) for correct integration. Clones giving the expected signals were purified to single cell clones and rescreened by PCR using the primers listed in Table 14. The final selected strains were called M845 (ptf1 deletion) and M843 (prp1/ptf1 deletion). The M843 strain underwent 5FOA plating to remove the pyr4/hygromycin marker and the resulting strain was named M1070.

TABLE 16
Primers for screening ptf1 integration and strain
purity.
For screening integration of pTTv372

| Primer | Sequence |
|---|---|
| T1047_3449_screen_5flk_fwd | AGTTCCCTGATGTGGACCTG |
| T1014_screen_5flk_pyr_rev | GGAGAATTTCGTGCGATCC |
| T1015_screen_3flk_hygro_fwd | GCATGGTTGCCTAGTGAATG |
| T1310_3449_screen_3flk_rev | TTTCATTGCCGCTCATCATA |

For screening deletion of ptf1 ORF

| Primer | Sequence |
|---|---|
| T1049_3449_orf_fwd | CTTATGGCGGAGAAGCTGAC |
| T1050_3449_orf_rev | GGTTTGCTAGCCGTTCAGAG |

Generation of ptf2 Deletion Plasmid

The deletion plasmid pTTv373 for the transcription factor ptJ2 (tre105269) was constructed essentially as described for pep1 deletion plasmid pTTv41 in WO/2013/102674, except that the marker used for selection was pyr4-hgh from pTTv194.

1042 bp of 5′ flanking region and 873 bp of 3′ flanking region were selected as the basis of the ptf2 deletion plasmid pTTv373. These fragments were amplified by PCR using the primers listed in Table 15. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Δpep4-pyr-hgh) with NotI digestion. To enable removal of the marker cassette, NotI restriction sites were introduced on both sides of the cassette. Vector backbone was EcoRI/XhoI digested pRS426 as in WO/2013/102674. The plasmid pTTv373 was constructed with the 5′ flank, 3′ flank, pyr4-hgh marker, and vector backbone using the yeast homologous recombination method described in WO/2013/102674. This deletion plasmid for ptf2 (pTTv372, Table 17) results in a deletion in the ptf2 locus and covers the complete coding sequence of PTF2.

TABLE 17
Primers for generating ptf2 deletion plasmids.
Deletion plasmid pTTv373 (Δptf1-pyr4-hgh), vector
backbone pRS426

| Primer | Sequence |
|---|---|
| T1311_105269_ | GTAACGCCAGGGTTTTCCCAGTCACGACGGTTT |
| 5flkfw_vector | AAACTAATTGACCCGGACAAGGAG |
| T1312_105269_ | GCGCTGGCAACGAGAGCAGAGCAGCAGTAGTCG |
| 5flkrev_pyr4Prom | ATGCTAGGCGGCCGCGAAGAACATGGGGAAGCA |
| | AA |
| T1313_105269_ | CAACCAGCCGCAGCCTCAGCCTCTCTCAGCCTC |
| 3flkfw_pyr4loop | ATCAGCCGCGGCCGCCCATTTCCTTTGGTTTGT |
| | GG |
| T1314_105269_ | GCGGATAACAATTTCACACAGGAAACAGCGTTT |
| 3flkrev_vector | AAACACATGGTCAATCCCCACAGT |

Generation of ptf2 Deletion Strain

To isolate the deletion cassette, plasmid pTTv373 was digested with Pinel and the correct fragment purified from an agarose gel using a QIAquick Gel Extraction Kit (Qiagen). Approximately 5 μg of the deletion cassette was used to transform the M788 pyr4-strain. Preparation of protoplasts and transformation were carried out essentially as described in WO/2013/102674 using hygromycin selection.

Transformants were picked as first streaks. Growing streaks were screened by PCR (using the primers listed in Table 18) for correct integration. Clones giving the expected signals were purified to single cell clones and rescreened by PCR using the primers listed in Table 16. The final strain was named M847.

TABLE 18
Primers for screening ptf2 integration and strain purity.
For screening integration of pTTv373

| Primer | Sequence |
|---|---|
| T1315_105269_screen_5flk_fwd | CTCACATTGCCTCGAACAGA |
| T1084_screen_5flk_pyr_rev | TCTTGAGCACGACAATCGAC |
| T1015_screen_3flk_hygro_fwd | GCATGGTTGCCTAGTGAATG |
| T1316_59740_screen_3flk_rev | ATCGTTGCACATCAGGTGAG |

For screening deletion of ptf2 ORF

| Primer | Sequence |
|---|---|
| T1317_105269_orf_fwd | AGTCCTACAGGGTGGCATTG |
| T1318_105269_orf_rev | TCGACATCGAGCATCAACTC |

Generation of ptf3 Deletion Plasmid

The deletion plasmid pTTv374 for the transcription factor ptf3 (tre59740) was constructed essentially as described for pep1 deletion plasmid pTTv41 in WO/2013/102674, except that the marker used for selection was pyr4-hgh from pTTv194.

994 bp of 5′ flanking region and 912 bp of 3′ flanking region were selected as the basis of the ptf3 deletion plasmid pTTv374. These fragments were amplified by PCR using the primers listed in Table 17. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Δpep4-pyr-hgh) with NotI digestion. To enable removal of the marker cassette, NotI restriction sites were introduced on both sides of the cassette. Vector backbone was EcoRI/XhoI digested pRS426 as in Example 1. The plasmid pTTv374 was constructed with the 5′ flank, 3′ flank, pyr4-hgh marker, and vector backbone using the yeast homologous recombination method described in WO/2013/102674. This deletion plasmid for ptf3 (pTTv374, Table 19) results in a deletion in the ptf3 locus and covers the complete coding sequence of PTF3.

TABLE 19
Primers for generating ptf3 deletion plasmids.
Deletion plasmid pTTv374 (Δptf3-pyr4-hgh), vector backbone pRS426

| Primer | Sequence |
|---|---|
| T1319_59740_5flkfw_vector | GTAACGCCAGGGTTTTCCCAGTCACGACGGTTTAAACCTCGAA |
| | AGCAGCCAACAAAT |
| T1320_59740_5flkrev_pyr4Prom | GCGCTGGCAACGAGAGCAGAGCAGCAGTAGTCGATGCTAGGC |
| | GGCCGCCGCTGGGCTTGATTAACATT |
| T1321_59740_3flkfw_pyr4loop | CAACCAGCCGCAGCCTCAGCCTCTCTCAGCCTCATCAGCCGCG |
| | GCCGCTGCTGCCAATTCATTTACCA |
| T1322_59740_3flkrev_vector | GCGGATAACAATTTCACACAGGAAACAGCGTTTAAACTTCCAG |
| | ATCCTCCTTTATAGCC |

Generation of ptf3 Deletion Strain

To isolate the deletion cassette, plasmid pTTv374 was digested with Pinel and the correct fragment purified from an agarose gel using a QIAquick Gel Extraction Kit (Qiagen). Approximately 5 μg of the deletion cassette was used to transform the M788 pyr4-strain. Preparation of protoplasts and transformation were carried out essentially as described in WO/2013/102674 using hygromycin selection.

Transformants were picked as first streaks. Growing streaks were screened by PCR (using the primers listed in Table 20) for correct integration. Clones giving the expected signals were purified to single cell clones and rescreened by PCR using the primers listed in Table 18. The final strain was named M958.

TABLE 20
Primers for screening ptf3 integration and strain purity.
For screening integration of pTTv374

| Primer | Sequence |
|---|---|
| T1323_59740_screen_5flk_fwd | CACCGCACGCTTCATACTTA |
| T1084_screen_5flk_pyr_rev | TCTTGAGCACGACAATCGAC |
| T1015_screen_3flk_hygro_fwd | GCATGGTTGCCTAGTGAATG |
| T1324_59740_screen_3flk_rev | AGCTAGAGGAGCGAGGGAAG |

For screening deletion of ptf3 ORF

| Primer | Sequence |
|---|---|
| T1325_59740_orf_fwd | AGCAAGTCACCTGGTTGGAC |
| T1326_59740_orf_rev | GAACGCGGTAGGTGATTGAT |

Generation of ptf4 Deletion Plasmid

The deletion plasmid pTTv461 for the transcription factor ptf4 (tre76505) was constructed essentially as described for pep1 deletion plasmid pTTv41 WO/2013/102674, except that the marker used for selection was pyr4-hgh from pTTv194.

1100 bp of 5′ flanking region and 1355 bp of 3′ flanking region were selected as the basis of the ptf4 deletion plasmid pTTv461. These fragments were amplified by PCR using the primers listed in Table 19. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Δpep4-pyr-hgh) with NotI digestion. To enable removal of the marker cassette, NotI restriction sites were introduced on both sides of the cassette. Vector backbone was EcoRI/XhoI digested pRS426 as in WO/2013/102674. The plasmid pTTv461 was constructed with the 5′ flank, 3′ flank, pyr4-hgh marker, and vector backbone using the yeast homologous recombination method described in WO/2013/102674. This deletion plasmid for ptf4 (pTTv461, Table 21) results in a deletion in the ptf4 locus and covers the complete coding sequence of PTF4.

TABLE 21
Primers for generating ptf4 deletion plasmids.
Deletion plasmid pTTv461 (Δptf4-pyr4-hgh), vector backbone pRS426

| Primer | Sequence |
|---|---|
| T1712_76505_5flkfw_vect | GTAACGCCAGGGTTTTCCCAGTCACGACGGTTTAAACGAGCAT |
| | TCGCTAGTGGGAAG |
| T1713_76505_5flkrev_pyr4Prom | GCGCTGGCAACGAGAGCAGAGCAGCAGTAGTCGATGCTAGGC |
| | GGCCGCTGGTAAGGGTGATGGAGGAG |
| T1714_76505_3flkfw_pyr4loop | CAACCAGCCGCAGCCTCAGCCTCTCTCAGCCTCATCAGCCGCG |
| | GCCGCCTCTCATACGCCTGACACGA |
| T1715_76505_3flkrev_vect | GCGGATAACAATTTCACACAGGAAACAGCGTTTAAACCGTTCT |
| | TTCCACCCAAGGTA |

Generation of ptf4 Deletion Strain

To isolate the deletion cassette, plasmid pTTv461 was digested with Pinel and the correct fragment purified from an agarose gel using a QIAquick Gel Extraction Kit (Qiagen). Approximately 5 μg of the deletion cassette was used to transform the M1070 pyr4-strain (with prp1/ptf1 deletions). Preparation of protoplasts and transformation were carried out essentially as described in WO/2013/102674 using hygromycin selection.

Transformants were picked as first streaks. Growing streaks were screened by PCR (using the primers listed in Table 22) for correct integration. Clones giving the expected signals were purified to single cell clones and rescreened by PCR using the primers listed in Table 22.

TABLE 22
Primers for screening ptf4 integration and strain purity.
For screening integration of pTTv461

| Primer | Sequence |
|---|---|
| T1716_76505_screen_5flkfw | TCTGGCTCTGACGTTGATTG |
| T1084_screen_5flk_pyr_rev | TCTTGAGCACGACAATCGAC |
| T1015_screen_3flk_hygro_fwd | GCATGGTTGCCTAGTGAATG |
| T1717_76505_screen_3flkrev | TCAAGCCTGTCAAACCGAAT |

For screening deletion of ptf4 ORF

| Primer | Sequence |
|---|---|
| T1718_76505_orf_fw | GACAGAAGAAGGACCGCAAG |
| T1719_76505_orf_rev | ATCTCTGAGGCGATCTGGAA |

Generation of ptf7 Deletion Plasmid

The deletion plasmid pTTv462 for the transcription factor ptf7 (tre106259) was constructed essentially as described for pep1 deletion plasmid pTTv41 in WO/2013/102674, except that the marker used for selection was pyr4-hgh from pTTv194.

971 bp of 5′ flanking region and 985 bp of 3′ flanking region were selected as the basis of the ptf7 deletion plasmid pTTv462. These fragments were amplified by PCR using the primers listed in Table 23. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Δpep4-pyr-hgh) with NotI digestion. To enable removal of the marker cassette, NotI restriction sites were introduced on both sides of the cassette. Vector backbone was EcoRI/XhoI digested pRS426 as in WO/2013/102674. The plasmid pTTv462 was constructed with the 5′ flank, 3′ flank, pyr4-hgh marker, and vector backbone using the yeast homologous recombination method described in WO/2013/102674. This deletion plasmid for ptf7 (pTTv462, Table 23) results in a deletion in the ptf7 locus and covers the complete coding sequence of PTF7.

Table 23. Primers for Generating ptf7 Deletion Plasmids.

Deletion plasmid pTTv462 (Δptf7-pyr4-hgh), vector backbone pRS426

| Primer | Sequence |
|---|---|
| T1704_106259_5flkfw_vect | GTAACGCCAGGGTTTTCCCAGTCACGACGGTTTAAACGGGCAA |
| | GGAGTTTCTTAGGG |
| T1705_106259_5flkrev_pyr4Prom | GCGCTGGCAACGAGAGCAGAGCAGCAGTAGTCGATGCTAGGC |
| | GGCCGCACATCGCATCTGACCTTCCT |
| T1706_106259_3flkfw_pyr4loop | CAACCAGCCGCAGCCTCAGCCTCTCTCAGCCTCATCAGCCGCG |
| | GCCGCTATCATGGCAGCTGATGCTC |
| T1707_106259_3flkrev_vect | GCGGATAACAATTTCACACAGGAAACAGCGTTTAAACGCGGTT |
| | TTCGTCTGTCTAGG |

Generation of ptf7 Deletion Strain

To isolate the deletion cassette, plasmid pTTv462 was digested with Pinel and the correct fragment purified from an agarose gel using a QIAquick Gel Extraction Kit (Qiagen). Approximately 5 μg of the deletion cassette was used to transform the M1070 pyr4-strain (with prp1/ptf1 deletions). Preparation of protoplasts and transformation were carried out essentially as described in WO/2013/102674 using hygromycin selection.

Transformants were picked as first streaks. Growing streaks were screened by PCR (using the primers listed in Table 24) for correct integration. Clones giving the expected signals were purified to single cell clones and rescreened by PCR using the primers listed in Table 22.

TABLE 24
Primers for screening ptf7 integration and strain purity.
For screening integration of pTTv462

| Primer | Sequence |
|---|---|
| T1708_106259_screen_5flk | ACAGCCACGAGACCTTTGAG |
| T1084_screen_5flk_pyr_rev | TCTTGAGCACGACAATCGAC |
| T1015_screen_3flk_hygro_fwd | GCATGGTTGCCTAGTGAATG |
| T1709_106259_screen_3flkrev | CATATCGCCCACTTCGTACA |

For screening deletion of ptf7 ORF

| Primer | Sequence |
|---|---|
| T1710_106259_orf_fw | AGACGTACATGCAGCACTCG |
| T1711_106259_orf_rev | CAGTCCCTCTCCCGGATACT |

Generation of ptf8 Deletion Plasmid

The deletion plasmid pTTv463 for the transcription factor ptf8 (tre106706) was constructed essentially as described for pep1 deletion plasmid pTTv41 in WO/2013/102674, except that the marker used for selection was pyr4-hgh from pTTv194.

999 bp of 5′ flanking region and 1038 bp of 3′ flanking region were selected as the basis of the ptf8 deletion plasmid pTTv463. These fragments were amplified by PCR using the primers listed in Table 25. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Δpep4-pyr-hgh) with NotI digestion. To enable removal of the marker cassette, NotI restriction sites were introduced on both sides of the cassette. Vector backbone was EcoRI/XhoI digested pRS426 as in WO/2013/102674. The plasmid pTTv463 was constructed with the 5′ flank, 3′ flank, pyr4-hgh marker, and vector backbone using the yeast homologous recombination method described in WO/2013/102674. This deletion plasmid for ptf8 (pTTv463, Table 25) results in a deletion in the ptf8 locus and covers the complete coding sequence of PTF8.

TABLE 25
Primers for generating Mg deletion plasmids.
Deletion plasmid pTTv463 (Δptf8-pyr4-hgh), vector backbone pRS426

| Primer | Sequence |
|---|---|
| T1696_106706_5flkfw_vect | GTAACGCCAGGGTTTTCCCAGTCACGACGGTTTAAACAAGAAG |
| | GCGCAAAGACGTTA |
| T1697_106706_5flkrev_pyr4Prom | GCGCTGGCAACGAGAGCAGAGCAGCAGTAGTCGATGCTAGGC |
| | GGCCGCTAGAAGAAAATCGGGCATCG |
| T1698_106706_3flkfw_pyr4loop | CAACCAGCCGCAGCCTCAGCCTCTCTCAGCCTCATCAGCCGCG |
| | GCCGCGGACGCCTTCTGTCCATAAA |
| T1699_106706_3flkrev_vect | GCGGATAACAATTTCACACAGGAAACAGCGTTTAAACAATTTC |
| | CCGCTGGTTTCTCT |

Generation of ptf8 Deletion Strain

To isolate the deletion cassette, plasmid pTTv463 was digested with Pinel and the correct fragment purified from an agarose gel using a QIAquick Gel Extraction Kit (Qiagen). Approximately 5 μg of the deletion cassette was used to transform the M1070 pyr4-strain (with prp1/ptf1 deletions). Preparation of protoplasts and transformation were carried out essentially as described in WO/2013/102674 using hygromycin selection.

Transformants were picked as first streaks. Growing streaks were screened by PCR (using the primers listed in Table 26) for correct integration. Clones giving the expected signals were purified to single cell clones and rescreened by PCR using the primers listed in Table 24.

TABLE 26
Primers for screening ptf8 integration and strain purity.
For screening integration of pTTv463

| Primer | Sequence |
|---|---|
| T1700_106706_screen_5flkfw | GTTTCTTGGAGACCCGTCAT |
| T1084_screen_5flk_pyr_rev | TCTTGAGCACGACAATCGAC |
| T1015_screen_3flk_hygro_fwd | GCATGGTTGCCTAGTGAATG |
| T1701_106706_screen_3flkrev | TGTCCAAGGTCGATGTCAAG |

For screening deletion of ptf8 ORF

| Primer | Sequence |
|---|---|
| T1702_106706_orf_fw | TGCGACTTTCGACATGAGTC |
| T1703_106706_orf_rev | GAAACTCAGACGCGTTAGGC |

Generation of ptf9 Deletion Plasmid

The deletion plasmid pTTv464 for the transcription factor ptf9 (tre108940) was constructed essentially as described for pep1 deletion plasmid pTTv41 in WO/2013/102674, except that the marker used for selection was pyr4-hgh from pTTv194.

1034 bp of 5′ flanking region and 1000 bp of 3′ flanking region were selected as the basis of the ptf9 deletion plasmid pTTv464. These fragments were amplified by PCR using the primers listed in Table 27. Template used in the PCR of the flanking regions was from the T. reesei wild type strain QM6a. The products were separated with agarose gel electrophoresis and the correct fragments were isolated from the gel with a gel extraction kit (Qiagen) using standard laboratory methods. The pyr4-hgh cassette was obtained from pTTv194 (Δpep4-pyr-hgh) with NotI digestion. To enable removal of the marker cassette, NotI restriction sites were introduced on both sides of the cassette. Vector backbone was EcoRI/XhoI digested pRS426 as in WO/2013/102674. The plasmid pTTv464 was constructed with the 5′ flank, 3′ flank, pyr4-hgh marker, and vector backbone using the yeast homologous recombination method described in WO/2013/102674. This deletion plasmid for ptf9 (pTTv464, Table 27) results in a deletion in the ptf9 locus and covers the complete coding sequence of PTF9.

TABLE 27
Primers for generating p09 deletion plasmids.
Deletion plasmid pTTv464 (Δptf9-pyr4-hgh), vector backbone pRS426

| Primer | Sequence |
|---|---|
| T1688_108940_5flkfw_vect | GTAACGCCAGGGTTTTCCCAGTCACGACGGTTTAAACAGCTGG |
| | AGACGCAATACCTG |
| T1689_108940_5flkrev_pyr4Prom | GCGCTGGCAACGAGAGCAGAGCAGCAGTAGTCGATGCTAGGC |
| | GGCCGCAGCATTCGCTCCGTGTAAGT |
| T1690_108940_3flkfw_pyr4loop | CAACCAGCCGCAGCCTCAGCCTCTCTCAGCCTCATCAGCCGCG |
| | GCCGCTTCTGTTTCCTCACGGCTCT |
| T1691_108940_3flkrev_vect | GCGGATAACAATTTCACACAGGAAACAGCGTTTAAACTACACC |
| | CCACACGAGAACAA |

Generation of ptf9 Deletion Strain

To isolate the deletion cassette, plasmid pTTv464 was digested with Pinel and the correct fragment purified from an agarose gel using a QIAquick Gel Extraction Kit (Qiagen). Approximately 5 μg of the deletion cassette was used to transform the M1070 pyr4-strain (with prp1/ptf1 deletions). Preparation of protoplasts and transformation were carried out essentially as described in WO/2013/102674 using hygromycin selection.

Transformants were picked as first streaks. Growing streaks were screened by PCR (using the primers listed in Table 28) for correct integration. Clones giving the expected signals were purified to single cell clones and rescreened by PCR using the primers listed in Table 26.

TABLE 28
Primers for screening ptf9 integration and strain purity.
For screening integration of pTTv464

| Primer | Sequence |
|---|---|
| T1692_108940_screen_5flkfw | CAAGGAGCCCAGCGTAATAG |
| T1084_screen_5flk_pyr_rev | TCTTGAGCACGACAATCGAC |
| T1015_screen_3flk_hygro_fwd | GCATGGTTGCCTAGTGAATG |
| T1693_108940_screen_3flkrev | CACCACGCTCATTCTCTTTG |

For screening deletion of ptf9 ORF

| Primer | Sequence |
|---|---|
| T1694_108940_orf_fw | CATTCGCCTCAAGCTTCACT |
| T1695_108940_orf_rev | GAGATCGGCCAGATCCTGT |

### Example 4. Cultivation of Single and Double Deletion Strains

Several regulatory proteins were identified that are likely involved in protease gene regulation. We created deletion strains for the prp1 (tre122069)/M675, ptf1 (tre3449)/M845, ptf2 (tre59740)/M847, ptf3 (tre59740)/M958, and the prp1/ptf1 double deletion strain M843 in the M577 interferon production strain. The prp1 deletion strain M675 the double deletion strain M843.

These strains and their parental strain M577 were cultivated in 24 well cultures and in the fermentor. In 24 well culture the strains were grown in TrMM plus 20 g/L spent grain extract and 40 g/L lactose, and 100 mM PIPPS at pH 4.5. The interferon production levels were measured via immunoblotting the 24 well culture supernatants diluted so that 0.2 μl was loaded per well in a 4-20% PAGE gel. The interferon was detected with anti-IFN antibody (Abeam #ab9386) diluted 1 μg/ml in TBST. The secondary was goat anti-mouse IRdye 680 conjugated antibody (Li-Cor #926-68070) diluted 1:30,000 in TBST. Detection was done by near infrared fluorescence (700 nm) using a Li-Cor Odyssey CLX imager. In these studies, the prp1/ptf1 deletion strains M843 produced more full length and carrier bound interferon than M577. Also, M675 was improved over the control on day 7 of the culture.

The M675 strain was cultivated in fermentor compared to its parental strain M577. The cultivation was done in TrMM plus 40 g/l lactose, 20 g/l spent grain extract, and 20 g/l whole spent grain at pH 4.5 with the temperature shifting from 28° to 22° at 48 h and grown for 5 days. The interferon concentration as determined by immunoblotting. The fermentor supernatants were diluted so that 0.1 μl was loaded into each well into a 4-20% PAGE gel Immunblotting was done to calculate the concentration compared to an interferon standard curve ranging from 400 to 25 ng. The interferon was detected with anti-IFN antibody (Abcam #ab9386) diluted 1 μg/ml in TBST. The secondary was goat anti-mouse IRdye 680 conjugated antibody (Li-Cor #926-68070) diluted 1:30,000 in TBST. Detection was done by near infrared fluorescence (700 nm) using a Li-Cor Odyssey CLX imager. Under these conditions the M675 strain could produce a maximum of 0.835 g/l on day 2 and 0.585 g/l on day 3. The maximum level produced by the M577 control was 500 mg/l. Thus the deletion of the regulatory protein resulted in a small improvement in interferon production.

The same strains were cultivated again with different media conditions. The cultivation was done in TrMM plus 20 g/l yeast extract, 40 g/l cellulose, 80 g/l cellobiose, and 40 g/l sorbose at pH 4.5 with the temperature shifting from 28° to 22° at 48 h and grown for 6 days. Again the M675 strain produced slightly better than the M577 control strain. On day 3 the M675 strain produced 1.05 g/l and on day 4 slightly more 1.2 g/l. These results can be compared to the M577 levels of 0.84 g/l on day 3 and 0.60 g/l on day 4. The tre122069 regulatory protein deletion strain produced more interferon than its parental strain. The increase was observed under two different media conditions when the strains were grown in the fermentor.

To confirm these results and compare all the strains the cultivations were done in TrMM plus 20 g/l yeast extract, 40 g/l cellulose, 80 g/l cellobiose, and 40 g/l sorbose at pH 4.5 with the temperature shifting from 28° to 22° at 48 h for 6 days.

The fermentor supernatants were diluted so that 0.1 μl was loaded into each well into a 4-20% PAGE gel Immunoblotting was done to calculate the concentration compared to an interferon standard curve ranging from 400 to 25 ng. The interferon was detected with anti-IFN antibody (Abeam #ab9386) diluted 1 μg/ml in TBST. The secondary was goat anti-mouse IRdye 680 conjugated antibody (Li-Cor #926-68070) diluted 1:30,000 in TBST. Detection was done by near infrared fluorescence (700 nm) using a Li-Cor Odyssey CLX imager.

The results can be seen in Table 29 [TABLE 29 IS MISSING??]. In agreement with previous cultivations the M675 strain outperformed M577 control strain. On day 3 the M577 strain produced 1.2 g/l and 0.9 g/L on day 4. The M675 produced 1.1 g/L on day 3 and 1.4 g/L on day 4. The peak production day shifted was delayed by one day after the prp1 deletion. The prp1/ptf1 double deletion strain M843 produced significantly more interferon than both control strains. On day 3 it was 1.5 g/L and on day 4 it produced 2.4 g/L. The double deletion strain improved the production level 2-fold compared to the parental M577.

The single deletions of ptf1 in M845 and ptf2 in M847 affected the growth rate of the strains, as seen from the CO2 production and the base and sugar consumption profiles observed during the cultivations. The M577, M675, and M843 grew at a similar pace, but the M845 and M847 strains with ptf1 and ptf2 single deletions grew more quickly. Under these batch conditions the faster growing strains were running out of nutrients sooner than the other three strains. A side effect of growing too well under these conditions was that the faster strains were not able to reach high production levels. The highest production level was 0.9 g/L on day 3. By day 4 the faster cultures appeared to have nearly exhausted their sugar supplies. The best slower growing strains produced their maximum amount of interferon on day 4. More optimization of the culture conditions would be needed to see the maximal production possible from the ptf1 and ptf2 single deletion strains.

The single deletion of ptf3 in M958 seemed to reduce the growth of the strain under the conditions the strain was tested. Interferon production in M958 was 0.64 g/L compared to the parental strain 1.2 g/L.

There seems to be a special relationship between ptf1 and prp1. This was previously observed while doing transient silencing studies with siRNAs, because when both genes were simultaneously silenced there was a larger effect than when they were silenced alone. Silencing of these factors simultaneously led to a large mRNA downregulation of several protease genes including pep8, pep9, pep11, cpa2, cpa3, and amp3. This was one reason why these two regulators were deleted from the same strain.

There appears to be a downregulation of proteases in the M843 strain. Protease activity against casein was tested using the EnzChek protease assay kit (Molecular probes #E6638, green fluorescent casein substrate). The working stock solution was prepared by diluting the stock to 10 μg/ml in 50 mM sodium citrate, pH 4.5. The culture supernatants were diluted with sodium citrate buffer so that the total protein concentration in each would be 0.65 mg/ml. 100 μl of the diluted substrate was combined with the diluted culture supernatants in a black 96 well sample plate. The plate was then covered and kept at 37° C. for one to three hours. Fluorescence readings were taken at one, two, and three hours with a Varioskan fluorescent plate reader (Thermo Scientific) using 485 nm excitation and 530 nm emission. Control wells with supernatant without substrate were used as background controls. The nonspecific background signal was subtracted from specific protease activity measurement. Clearly the M843 double deletion strain generated less protease activity along the whole time course of the cultivation compared to M577 (Table 30). Between day 4 and 6, there was around 33% less activity. The reduced protease activity is the likely cause of the 2-fold increase in interferon production seen with the M843 strain.

TABLE 29
Interferon immunoblot data from Triab137-141 cultivations with M577,
M675, M843, M845, and M847 production strains. Two separate blots
were used to quantify all the samples. The strains were grown in
TrMM plus 20 g/l yeast extract, 40 g/l cellulose, 80 g/l cellobiose,
and 40 g/l sorbose at pH 4.5. The interferon in 0.1 μl of supernatant
was detected with anti-IFN antibody (Abcam #ab9386) diluted 1
μg/ml in TBST. The secondary was goat anti-mouse IRdye 680 conjugated
antibody (Li-Cor # 926-68070) diluted 1:30,000 in TBST. Detection
was done by near infrared fluorescence (700 nm).

| | M577 #1 | M675 | M843 | M577 #2 | M845 | M847 |
|---|---|---|---|---|---|---|
| | g/L | g/L | g/L | g/L | g/L | g/L |
| day 2 | 0.4 | 0.8 | 0.4 | 0.5 | 0.4 | 0.6 |
| day 3 | 1.2 | 1.1 | 1.5 | 1.1 | 0.9 | 0.9 |
| day 4 | 0.9 | 1.4 | 2.4 | 0.8 | 0.3 | 0.4 |
| day 5 | 0.3 | 0.3 | 0.5 | 0.4 | 0.2 | 0.3 |

TABLE 30
Total protease activity against fluorescent casein substrate. All
supernatant samples were normalized to 0.65 mg/ml of total protein
before adding casein substrate. The substrate was incubated in the
supernatant at 37° C. for 2 hours. Fluorescence was
measured using 480 nm excitation and 530 nm emission.

| fluorescent units | M577 | M675 | M843 | M845 | M847 |
|---|---|---|---|---|---|
| day 2 | 3.8 | 3.8 | 4.6 | 3.0 | 6.5 |
| day 3 | 5.0 | 4.8 | 4.3 | 6.9 | 7.1 |
| day 4 | 7.4 | 5.2 | 5.1 | 10.5 | 7.9 |
| day 5 | 12.1 | 12.2 | 8.4 | 10.1 | 12.7 |
| day 6 | 15.2 | 16.4 | 10.0 | 13.6 | 18.2 |

The regulator deletions have been made into the M577 strain where there were already 8 proteases deleted from the strain. Thus, the full benefit of the prp1/ptf1 deletion could not been seen. In the future the deletions will be made into the wild type background strain with a full set of proteases. This will reveal the full potential of the double regulator deletions.

Interestingly, prp1 (tre122069) is not a transcription factor, but it is classified as a BTB/POZ regulatory protein. A general property of the BTB domain is to mediate homomeric dimerization and is involved in heteromeric interactions with a number of proteins. BTB/POZ domains from several zinc finger proteins have been shown to mediate transcriptional repression and to interact with components of histone deacetylase co-repressor complexes. It is believed that histone deacetylase in the BTB protein complex modifies the chromatin structure needed for transcriptional repression. However, there are also examples where the BTB domain is capable of mediating transcriptional activation as well. The ptf1 is a more standard fungal transcription factor. The N-terminal region of the protein contains a Cys-rich motif that is involved in zinc-dependent binding of DNA.

### Example 5. Silencing Protease Regulators in the M843 Interferon Production Strain

Seeking to further improve the M843 strain, 10 candidate transcription factors and regulatory proteins were transiently silenced in 24 well cultures and improvement of interferon production was monitored via immunoblotting. The siRNAs were prepared as described in Example 1. These siRNA/lipid carrier complexes were added directly to 24 well cultures of M843 and dosed daily up to six days in culture. Two wells were used per treatment. Lipid only treatment was done as the mock control. The Trichoderma cultures were started with 1×106 spores/well and grown in TrMM with 20 g/L spent grain extract and 40 g/L lactose, pH 4.5. The siRNAs were added so that the final concentration in the 3 ml cultures was 200 nM. The siRNAs were added each day up to day 6 of the cultures. The siRNAs are listed in Table 31.

TABLE 31
siRNA sequences for transcription factor and
regulatory proteins.

| Gene abbrev- | | |
|---|---|---|
| iation | siRNA name | siRNA sequence |
| ptf2 | TRIRE105269 | CUCAUGUUGGCUGACGGAATT |
| ptf2 | TRIRE105269_2 | GCAAUCUCGCCGGCUCAAUTT |
| ptf2 | TRIRE105269_3 | GCAAGAUGCUUCCGUCACUTT |
| ptf3 | TRIRE59740 | GGUGCACGUUGCUGCCAAUTT |
| ptf3 | TRIRE59740_2 | CACGUUGCUGCCAAUUCAUTT |
| ptf3 | TRIRE59740_3 | GGUUGGACGUGGUCAUGUUTT |
| ptf4 | TRIRE76505 | GGCAAGAAGUUCUCUCGCATT |
| ptf4 | TRIRE76505_2 | CUGUGUAUCUCCAGUCCCATT |
| ptf4 | TRIRE76505_3 | CCCUUUGAGUGCAACGAGUTT |
| ptf5 | TRIRE103158 | GUCAAGAUGACGAUGAAAUTT |
| ptf5 | TRIRE103158_2 | CUGACAUCAAUCGGCGUAATT |
| ptf5 | TRIRE103158_3 | GAGCAAUUGCGGCACAGGATT |
| ptf6 | TRIRE103275 | CAAAGAAACGCUCCAGAAATT |
| ptf6 | TRIRE103275_2 | GAAACUCACACACAUGUUUTT |
| ptf6 | TRIRE103275_3 | CUCAUGAAGGCCAUCCAGATT |
| ptf7 | TRIRE106259 | CAGAUGGUCCUCACACAAUTT |
| ptf7 | TRIRE106259_2 | GUCUGGUGGUGCUGCUUCATT |
| ptf7 | TRIRE106259_3 | GGCAAGACGUGGUUCUGGATT |
| ptf8 | TRIRE106706 | GUUCAGUCUGCGUACGAUUTT |
| ptf8 | TRIRE106706_2 | CCGUAUUCCCAUCAUCAUATT |
| ptf8 | TRIRE106706_3 | CUUCAACUUGCUUAUGAAATT |
| ptf9 | TRIRE108940 | GAAGGAACCGCGAUUCAUUTT |
| ptf9 | TRIRE108940_2 | GAUUCAUUACUUCGAUCUUTT |
| ptf9 | TRIRE108940_3 | GUGCAUUGUGCUUCAUCUATT |
| prp2 | TRIRE102947 | GCGUCAAGGCCCUCGGCCATT |
| prp2 | TRIRE102947_2 | GCCAUCUCGCGACCCAGAATT |
| prp2 | TRIRE102947_3 | CCUUCAUGAUUACUGCCAATT |

The cultures were sampled on day 3, 4, 5, 6, and 7 and prepared for immunblotting with a rabbit anti-interferon alpha 2 b antibody (Abeam # ab9386). The interferon primary antibody was diluted to 1 μg/ml in TBST and incubated for 1 hour shaking at room temperature. A control mouse antibody against CBHI (mab261, VTT) was diluted 1:20,000 in TBST and incubated shaking at room temperature for 1 hour. The goat anti-rabbit secondary IRDye 680 (Li-Cor #926-68070) and goat anti-mouse IRDye 800 (Li-Cor #926-32210) secondary antibodies were diluted 1:30,000 in TBST and incubated with the blots for 45 minutes at room temperature shaking. The blots were washed with TBST for 1 hour before scanning at 700 and 800 nm.

Analyzing the samples from day 4, it was seen that all of the transcription factor treatments improved the production level of interferon. The ptf4, ptf6, ptf8, and ptf9 siRNA treatments resulted in production level improvements reaching over 2-fold (Table 32).

TABLE 32
Relative interferon expression levels measured from day 4 samples
taken from the siRNA treated 24 well cultures.

| siRNA | interferon | fold |
|---|---|---|
| treatment | (fluorescent units) | improvement |
| ptf2 | 653 | 1.3 |
| ptf3 | 788 | 1.6 |
| ptf4 | 1140 | 2.3 |
| ptf5 | 958 | 2.0 |
| ptf6 | 1175 | 2.4 |
| ptf7 | 764 | 1.6 |
| ptf8 | 1171 | 2.4 |
| ptf9 | 1334 | 2.7 |
| ptf10 | 714 | 1.5 |
| prp2 | 560 | 1.1 |
| Control | 489 | 1.0 |

The day 7 samples were similarly analyzed via immunoblotting to detect interferon expression and the secreted cellulase enzyme CBHI as a control. CBHI is the main secreted enzyme in Trichoderma reesei. Again all the treatments increased interferon expression over the mock control level (Table 33). The best treatments came from the ptf4, ptf6, ptf7, ptf8, and ptf10 siRNAs. Compared to the day 4 treatment results, the ptf4, ptf6, and ptf8 were again among the best again on day 7. Normalizing the results according to the CBHI expression level the ptf4, ptf6, ptf7, and ptf8 were the best improvements.

TABLE 33
Relative interferon and CBHI expression levels measured from day
7 samples taken from the siRNA treated 24 well cultures.

| | | | fold | |
|---|---|---|---|---|
| siRNA | interferon | CBHI | improvement- | IFN/ |
| treatment | (fluorescent units) | (fluorescent units) | interferon | CBHI |
| ptf2 | 827 | 1525 | 1.4 | 0.54 |
| ptf3 | 850 | 1355 | 1.5 | 0.62 |
| ptf4 | 991 | 1320 | 1.7 | 0.66 |
| ptf5 | 673 | 932 | 1.2 | 0.62 |
| ptf6 | 892 | 1325 | 1.6 | 0.67 |
| ptf7 | 1028 | 1370 | 1.8 | 0.75 |
| ptf8 | 911 | 1305 | 1.6 | 0.69 |
| ptf9 | 703 | 1305 | 1.2 | 0.53 |
| ptf10 | 904 | 1395 | 1.6 | 0.65 |
| prp2 | 602 | 1305 | 1.1 | 0.46 |
| Control | 571 | 1235 | 1.0 | 0.46 |

### Example 6. Cultivation of Strains with 3 Regulatory Protein Deletions in an Interferon Production Strain

The M843 strain where the protease regulatory protein prp1 and the protease transcription factor ptf1 have been deleted, went through marker loop-out, and the resulting pyr4-strain was designated M1070. The M1070 strain was used to produce new transcription factor deletions. Transient silencing experiments were conducted on M843 to evaluate whether additional transcription factors could improve interferon expression. The transcription factors ptf4 (tre76505), ptf7 (tre106259), ptf8 (tre106706), and ptf9 (tre108940) were among the best candidates. Deletion strains were prepared for these genes (as described in Example 3) in hope the deletions reduce protease activity and lead to higher interferon production levels. We were unable to get clean transformants for the ptf9 deletion in the interferon production strain.

The positive transformants from ptf4, ptf7, and ptf8 deletion strains were then cultured in 24 wells along with the M577 and M843 control interferon production strains. Two wells were used for each strain or transformant. In 24 well cultures the strains were grown in TrMM with diammonium citrate without ammonium sulfate, 100 mM PIPPS, 20 g/L spent grain extract, 40 g/L lactose at pH 4.5, shaking at 28° C. The supernatants were diluted so that 0.2 μl was loaded into each well into a 4-20% gel Immunoblotting was done to calculate the concentration compared to an interferon standard curve (200, 100, 50, and 25 ng). The interferon was detected with anti-IFN antibody (Abeam #ab9386) diluted 1 μg/ml in TBST. The secondary was goat anti-mouse IRdye 680 conjugated antibody (Li-Cor #926-68070) diluted 1:30,000 in TBST. Detection was done by near infrared fluorescence (700 nm).

The expression results from day 6 of the culture can be seen in Table 34. The original M577 strain contained 8 protease deletions and produced interferon at levels around 163 mg/L in this culture on day 6. The M843 strain was generated from deleting the ptf1 transcription factor and the prp1 regulatory protein. The resulting strain, as previously reported, produced 1.8-fold more interferon at 291 mg/L. This effect seems to be related to down-regulation of proteases. Starting from M843 strain we have now have separately deleted 3 other transcription factors. The best ptf4 transformant (461-13F) produced 1.5 times more interferon at a level of 401 mg/L. The top ptf7 transformant (462-18B) expressed also 1.5 times more at 450 mg/L. The overall best deletion was from ptf8, from which the best transformant gave 2 times more interferon than M843 and reached on overall level of 592 mg/L. Overall we have achieved an improvement from 162 mg/L to 592 mg/L by deleting 3 protease regulators. That is a 3.7-fold improvement. We do not yet know if the ptf4, ptf7, or ptf8 deletions have reduced the protease activity. We were unable to measure protease activity from these strains, because there are already 8 proteases deleted from the M577 strain. Future studies on the specific proteases affected by these regulators will be conducted.

TABLE 34
Immunoblot results detecting interferon expression from 24 well cultures
of transcription factor deletion strains for ptf4, ptf7, ptf8 and controls
M577 and M843. Two immunoblots were used to measure the interferon
concentration from all the samples. Control strains and standards were
included on both blots. A standard curve of 200, 100, 50, 25 ng of
interferon was used to calculate the culture concentration. Cultures
were grown in TrMM with diammonium citrate without ammonium
sulfate, 100 mM PIPPS, 20 g/L spent grain extract, 40 g/L lactose
at pH 4.5, shaking at 28° C. Immunoblot of interferon alpha
2b expression was made from 0.2 μl culture supernantat on
day 6 of culture.

| | interferon |
|---|---|
| Strain | mg/L |
| M577 #1 | 163 |
| M843 #1 | 266 |
| Δptf4-13B | 387 |
| Δptf4-13F | 401 |
| M577 #2 | 162 |
| M843 #2 | 291 |
| Δptf7-18A | 345 |
| Δptf7-18B | 450 |
| Δptf8-11A | 539 |
| Δptf8-11B | 592 |

### Example 7. Construction of RNAi Silencing Vectors for Protease Regulators and Protease Genes

RNAi silencing vectors are designed to contain target sequence in a single RNA hairpin molecule for multiple transcriptional regulator genes or a combination of transcriptional regulators and proteases genes.

The RNAi base vector pTTv436 contains a gpdA promoter to drive the expression of the hairpin RNA, sequence for targeted integration into the xylanase 1 locus (tre74223), and a pyr4/hygromycin loop-out marker. The 150-250 bp target sequence for each gene, intron loop, and cloning sequence containing DNA fragment can be synthesized by commercial providers. The artificially synthesised vector including the sense target sequence is digested with PmeI to release the target sequence fragment. The pTTv436 RNAi base vector is opened with FseI in the first step and the PmeI digested sense target sequence fragments is incorporated into the vector using yeast recombination cloning. The newly made pTTv436+sense target sequence vector is then opened with AscI. The antisense fragment is generated by PCR. The newly created antisense target sequence fragment is then combined into the AscI linearized vector using yeast recombination. The newly created vector expresses an RNA hairpin that is used for silencing all genes included in the construct.

### A Protease Regulatory Gene Silencing Construct

The antisense fragment is created by PCR with the following primers:

antisense_rev_prp2_ascI_loop
GAGAGAAGGGCACGTACTTACAAACACATCTCTTGCATAGGGCGCGCCCT
GGAGTGTCTTGATCATCA
antisense fw_prp1_asisI_vect
GTCAAGCTGTTTGATGATTTCAGTAACGTTAAGTGGATCCGCGATCGCGG
TGACCTGTTTACTCAGTT

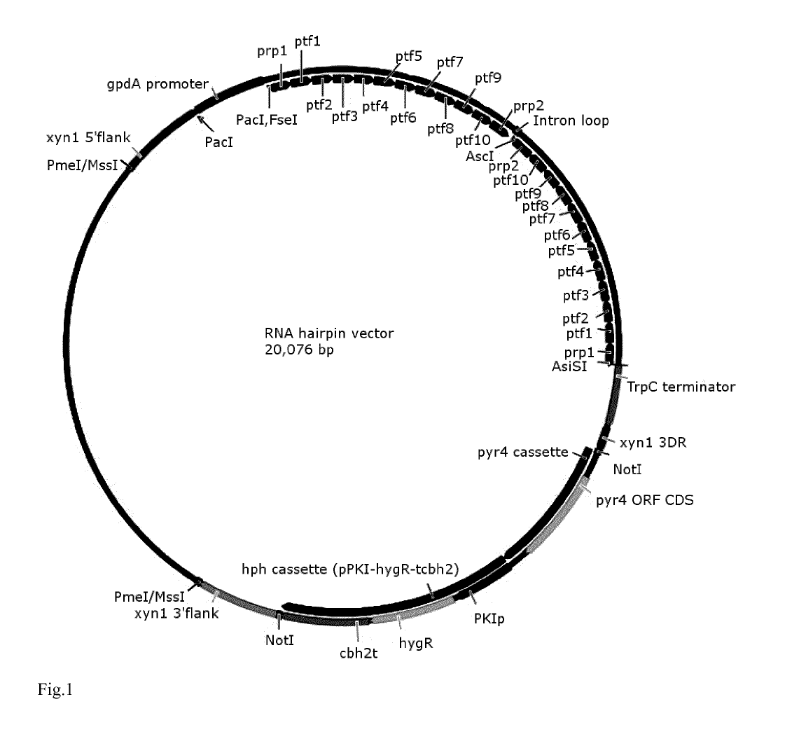

The following DNA fragment may be purchased from commercial sources to make the first part of the vector as described above. It contains 150 bp target sequence for 12 protease regulatory genes and an intron loop needed for hairpin formation. The silencing vector is constructed as described above with the pTTv436 RNAi base vector, target sequence DNA fragment, and PCR created antisense fragment. The final vector is shown in FIG. 1. The siRNA sequence of FIG. 1 is disclosed in SEQ ID NO: 131.

### Multiple Protease and Regulator Gene Silencing Vectors

Silencing vectors containing target sequences for protease genes and protease regulators were created in the same general way as described above with the pTTv436 RNAi base vector, target sequence DNA fragment, and PCR created antisense fragment. Three silencing vectors were made with proteases and regulators: small and large combination vectors with protease regulator genes and the third one, carboxypeptidase silencing vector, which can be constructed to include the regulatory genes.

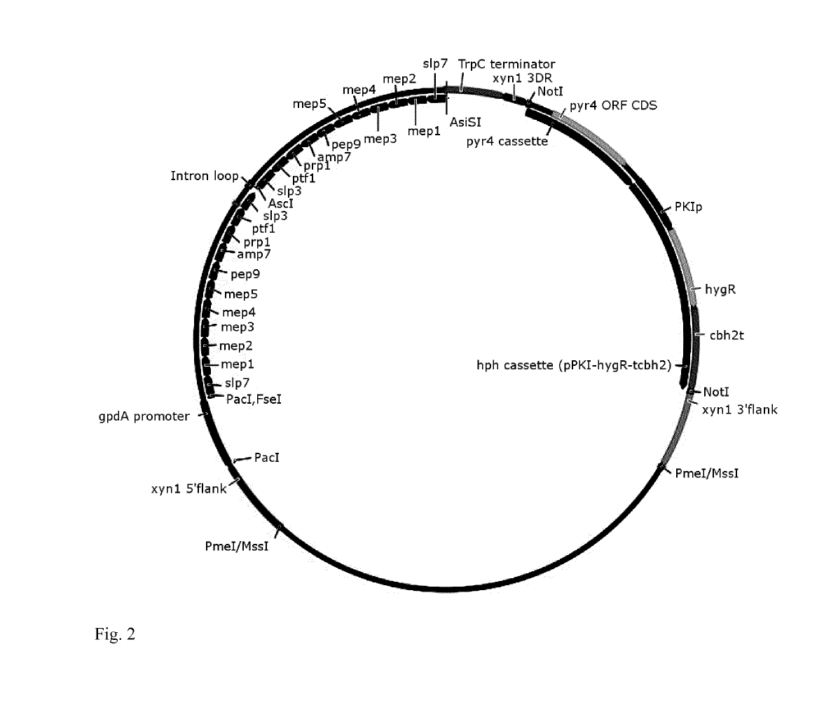

The small combination RNAi vector contains the following sequence and was synthesized by a commercial provider. It contains 250 bp target sequences for 9 protease and 2 regulatory proteins. The vector map can be seen in FIG. 2 and the DNA fragment sequence is shown in SEQ ID NO:132.

The large combination RNAi vector contains the following sequence and was synthesized by a commercial provider. It contains 150 bp target sequences for the following 19 protease slp7, tpp1, mep1, mep2, tre4308, mep4, mep5, pep8, pep9, amp7, lap4, tre66608, tre81115, tre111694, tre21659, tre21668, tre77577, tre23475, prp1, ptf1, slp3 and 2 regulatory proteins (prp1 and ptf1). The DNA fragment sequence is shown in SEQ ID NO:133.

The carboxypeptidase RNAi vector contains the following sequence of SEQ ID NO:134 and was synthesized by a commercial provider. It contains 150 bp target sequences for 7 carboxypeptidases. A further variant of this RNAi vector can include protease regulator target sequences for prp1 (tre122069) and ptf1 (tre3449) to create combination vectors as done with the small and large protease and regulator vectors described above.

The synthesised vectors including the sense target sequence were digested with PmeI to release the fragment. The pTTv436 RNAi base vector was opened with FseI in the first step and the PmeI digested sense target sequence fragments were incorporated into the vector using yeast recombination cloning. The newly made pTTv436+sense RNAi target sequence fragment vector was then reopened with AscI.

The RNA antisense fragments were created by PCR using the following primers::

T1615_antisense_rev_slp3_ascI_loop
GAGAGAAGGGCACGTACTTACAAACACATCTCTTGCATAGGGCGCGCCTT
CAGGGTGGTTGCGAAGAG
T1772_antisense_fw_slp7_asisI_vect
GTCAAGCTGTTTGATGATTTCAGTAACGTTAAGTGGATCCGCGATCGCCC
GTTACCTTGGCCGCGAAG

The carboxypeptidase antisense fragment was created by PCR using the primers:

T1773_antisense_rev_cp2_ascI_loop
CCTCGATATGAAAGTCTTCACCGCTGACTTGCTGCTCTAGGGCGCGCCCGT
CTACGTTGACCACGGGG
T1774_antisense_fw_cp3_asisI_vect
GTCAAGCTGTTTGATGATTTCAGTAACGTTAAGTGGATCCGCGATCGCTTT
AGCGGTGGCTCAACTGC

The newly created antisense target sequence fragments were then combined into the AscI linearized vector using yeast recombination. The final RNAi silencing vectors may be transformed into T. reesei strains to provide gene silencing of multiple protease and protease regulator genes.

siRNA sequences used in the expression vectors for the regulatory proteins and proteases are further disclosed in SEQ ID NOs:135-186.

1-21. (canceled)

22. A Trichoderma cell having reduced or no activity in one or more regulatory proteins selected from the group consisting of ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), ptf7 (SEQ ID NO: 11) and prp2 (SEQ ID NO:12).

23. The Trichoderma cell of claim 22, which is Trichoderma reesei.

24. The Trichoderma cell of claim 22, comprising a mutation in at least one gene encoding said regulatory protein selected from prp1, prp2, ptf1, ptf2, ptf3, ptf4, ptf5, ptf6, ptf7, ptf8, ptf9 and ptf10, said mutation rendering said regulatory protein non-functional.

25. The Trichoderma cell of claim 22, which is selected from the group consisting of Δprp1, Δptf1, Δprp1 Δptf1, Δptf2, Δptf3, Δptf4, Δptf4 Δprp1 Δptf1, App, Δptf7 Δprp1 Δptf1, Δptf9, Δptf9 Δprp1 Δptf1, Δptf8 and Δptf8 Δprp1 Δptf1 deletion mutant Trichoderma cells.

26. The Trichoderma cell of claim 22, comprising a mutation in at least one gene encoding a protease, said mutation reducing or eliminating the corresponding protease activity, and said protease being selected from the group consisting of pep 1, tsp1, slp1, gap1, gap2, pep4, pep3, and pep5.

27. The Trichoderma cell of claim 22, comprising a mutation in at least one gene encoding a protease, said mutation reducing or eliminating the corresponding protease activity, and said protease being selected from the group consisting of the following protease pep4, pep8, pep9, pep 11, slp5, cpa5, cpa2, cpa3, amp3, tpp1, pep12, amp2, mp1, mp2, mp3, mp4, mp5, amp1, sep1, slp2, slp3, slp6, slp7, and slp8.

28. The Trichoderma cell of claim 22, comprising a mutation in a gene encoding ALG3, wherein said mutation reduces or eliminates the corresponding activity.

29. The Trichoderma cell of claim 22, further comprising a first polynucleotide encoding the N-acetylglucosaminyltransferase I catalytic domain.

30. The Trichoderma cell of claim 22, further comprising a polynucleotide encoding an α-1,2-mannosidase, a mannosidase II, a galactosyl transferase and/or GDP-fucose synthesis activity.

31. The Trichoderma cell of claim 22, further comprising a recombinant nucleic acid encoding a heterologous mammalian polypeptide.

32. The Trichoderma cell of claim 31, wherein the mammalian polypeptide is glycosylated.

33. The Trichoderma cell of claim 31, wherein the mammalian polypeptide is selected from the group consisting of an antibody and their antigen-binding fragments, a growth factor, an interferon, a cytokine, and an interleukin.

34. A method of improving heterologous polypeptide production in a Trichoderma cell expression system, comprising
a) providing a Trichoderma cell according to claim 22 in which one or more regulatory proteins have reduced or eliminated activity, and
b) culturing said Trichoderma cell for production of a heterologous polypeptide, wherein the heterologous polypeptide is produced at a higher yield when compared to the heterologous polypeptide produced in a corresponding parental Trichoderma cell in which said one or more regulatory proteins do not have reduced or eliminated activity.

35. A method of making a heterologous polypeptide, comprising
a) providing a Trichoderma cell according to claim 22;
b) culturing said Trichoderma cell for production and secretion of a heterologous polypeptide in the culture medium; and,
c) recovering the heterologous polypeptide from the culture medium.

36. The method of claim 35, wherein the expression is reduced by contacting the cell with siRNA compounds directed against one or more of the genes encoding said regulatory proteins, wherein siRNA compound is directed against a gene encoding a regulatory protein selected from the group consisting of ptf1 (SEQ ID NO:1), prp1 (SEQ ID NO:2), ptf9 (SEQ ID NO:3), ptf3 (SEQ ID NO:4), ptf8 (SEQ ID NO:5), ptf5 (SEQ ID NO:6), ptf6 (SEQ ID NO:7), ptf2 (SEQ ID NO:8), ptf4 (SEQ ID NO:9), ptf10 (SEQ ID NO:10), ptf7 (SEQ ID NO:11) and prp2 (SEQ ID NO:12).

37. The method of claim 36, wherein the siRNA compound is further directed to a gene encoding a protease.

38. The method of claim 37, wherein said protease is selected from the group consisting of pep4, pep8, pep9, pep 11, slp5, cpa5, cpa2, cpa3, amp3, tpp1, pep12, amp2, mp1, mp2, mp3, mp4, mp5, amp1, sep1, slp2, slp3, slp6, slp7, slp8.

39. The Trichoderma cell of claim 26, comprising a deletion mutation in at least one gene encoding said regulatory protein selected from prp1, prp2, ptf1, ptf2, ptf3, ptf4, ptf5, ptf6, ptf7, ptf8, ptf9 and ptf10, said deletion mutation rendering said regulatory protein non-functional.

40. The Trichoderma cell of claim 26, comprising a deletion mutation in at least one gene encoding a protease, said mutation eliminating the corresponding protease activity.

41. The Trichoderma cell of claim 29, further comprising a second polynucleotide encoding the N-acetylglucosaminyltransferase II catalytic domain.
